(19) United States
(12) Patent Application Publication
(10) Pub. No.: US 2024/0124558 A1
(43) Pub. Date: Apr. 18, 2024

(54) ANTIBODIES BINDING TO SARS-COV-2 VIRUS AND USES THEREOF

(71) Applicant: ASSURE TECH. (HANGZHOU) CO., LTD., Hangzhou (CN)

(72) Inventors: Yiding CHEN, Beijing (CN); Xiangxi WANG, Beijing (CN); Ling ZHU, Beijing (CN); Yinan JIANG, Beijing (CN); Jingyun MIAO, Beijing (CN); Lili QIN, Beijing (CN); Pingju GE, Beijing (CN)

(73) Assignee: ASSURE TECH. (HANGZHOU) CO., LTD., Hangzhou (CN)

(21) Appl. No.: 18/391,559

(22) Filed: Dec. 20, 2023

Related U.S. Application Data
(63) Continuation of application No. PCT/CN2022/081623, filed on Mar. 18, 2022.

(30) Foreign Application Priority Data
Dec. 14, 2021 (CN) 202111529152.7

(51) Int. Cl. C07K 16/10; A61K 47/68; A61K 49/00; A61K 51/10; A61P 31/14

(52) U.S. Cl. CPC C07K 16/1003; A61K 47/6841; A61K 49/0058; A61K 51/1096; A61P 31/14; C07K 2317/21; C07K 2317/24; C07K 2317/622

(57) ABSTRACT

The embodiments of the present disclosure provide an antibody or antigen-binding fragment against SARS-CoV-2 spike(S) protein, comprising: three complementarity determining regions (HCDRs) of a heavy chain variable region or one or more variants thereof, the heavy chain variable region set forth as SEQ ID NO. 30 or SEQ ID NO. 46, each of the one or more variants having at most two amino acid changes compared to the corresponding CDR; and three complementarity determining regions (LCDRs) of a light chain variable region or one or more variants thereof, the light chain variable region set forth as SEQ ID NO. 32 or SEQ ID NO. 48, each of the one or more variants having at most two amino acid changes compared to the corresponding CDR.

### CROSS-REFERENCE TO RELATED APPLICATION

This application is a Continuation of International Application NO. PCT/CN2022/081623, filed on Mar. 18, 2022, which claims priority of Chinese Application No. 202111529152.7, filed on Dec. 14, 2021, the entire contents of each of which are hereby incorporated by reference.

### SEQUENCE LISTING

The instant application contains a Sequence Listing which has been submitted electronically in XML format and is hereby incorporated by reference in its entirety. The XML copy, created on Dec. 15, 2023, is named “20771-D005US00-Sequence Listing” and is 200,289 bytes in size.

### TECHNICAL FIELD

The present disclosure relates to the field of antibody technology, and in particular, to a neutralizing antibody that can bind to the SARS-CoV-2 virus and a use thereof.

### BACKGROUND

The coronavirus is a virus discovered in 2019 and named Severe Acute Respiratory Syndrome Coronavirus 2 (SARS-CoV-2) by WHO, which can cause viral pneumonia or lung infection in humans. The virus has so far caused about 240 million newly confirmed infections worldwide, resulting in about 4.8 million deaths. Common symptoms of coronavirus infection include fever, fatigue, and dry cough. Some patients may also experience symptoms related to the upper respiratory tract and digestive system, such as nasal congestion, runny nose, and diarrhea. In severe cases, patients may develop dyspnea (shortness of breath) after approximately one week. Rapid progression of the disease can lead to complications such as acute respiratory distress syndrome, septic shock, uncorrectable metabolic acidosis, and coagulation dysfunction. While some therapeutic drugs have been reported, further clinical trials are required to confirm their effectiveness. Additionally, research and development efforts are underway for vaccines, but it will take time before they can be clinically applied.

The new coronavirus belongs to the genus p of coronaviruses, and has an envelope. The viral particles are typically circular or elliptical in shape, often exhibiting polymorphism, with a diameter of 60-140 nm. The coronavirus shares a 70% homology with the severe acute respiratory syndrome virus (SARS-CoV), with the main sequence differences being found in the critical spike gene responsible for encoding the S-protein. This S-protein is crucial for the interaction between the virus and the host cell. Therefore, the development of antibodies against S-protein (especially active neutralizing antibodies) is the key for the prevention and/or treatment of this disease. For this reason, this application is proposed.

### SUMMARY

In view of the above-mentioned technical problems, the present disclosure provides a series of neutralizing antibodies or antigen-binding fragments that can bind to the SARS-CoV-2 virus, so as to achieve the following objectives of the disclosure.

The first purpose of the present disclosure is to provide an antibody or antigen-binding fragment against SARS-CoV-2 spike(S) protein;

The second purpose of the present disclosure is to provide a polynucleotide encoding the above-mentioned antibody or antigen-binding fragment, a corresponding recombinant vector, a host cell, a kit or pharmaceutical composition containing the antibody or antigen-binding fragment, etc.

The third purpose of the present disclosure is to provide a use of the above-mentioned antibody or antigen-binding fragment in the prevention or treatment of the coronavirus;

The fourth purpose of the present disclosure is to provide a use of the above-mentioned antibody or antigen-binding fragment in the neutralizing antibody detection, screening, purification, and preparation;

The fifth purpose of the present disclosure is to provide a method for preparing a neutralizing antibody or an antigen-binding fragment capable of binding to the SARS-CoV-2 virus;

The sixth purpose of the present disclosure is to provide a method for preventing or treating COVID-19.

In order to achieve the above purpose, the present disclosure adopts the following technical solutions.

The first aspect of the present disclosure provides an antibody or an antigen-binding fragment comprising three complementarity determining regions (HCDRs) of a heavy chain variable region or one or more variants thereof, the heavy chain variable region set forth as SEQ ID NO. 30 or SEQ ID NO. 46, each of the one or more variants having at most two amino acid changes compared to the corresponding CDR; and three complementarity determining regions (LCDRs) of a light chain variable region or one or more variants thereof, the light chain variable region set forth as SEQ ID NO. 32 or SEQ ID NO. 48, each of the one or more variants having at most two amino acid changes compared to the corresponding CDR.

Further, the HCDRs and LCDRs are numbered by a Kabat numbering scheme, and the antibody or the antigen-binding fragments includes:
i. HCDR1 set forth as SEQ ID NO. 103 or 127 or a variant thereof, wherein, the variant has at most two amino acid changes compared to the HCDR1;
ii. HCDR2 set forth as SEQ ID NO. 104 or 128 or a variant thereof, wherein the variant has at most two amino acid changes compared to the HCDR2;
iii. HCDR3 set forth as SEQ ID NO. 105 or 129 or a variant thereof, wherein the variant has at most two amino acid changes compared to the HCDR3;
iv. LCDR1 set forth as SEQ ID NO. 106 or 130 or a variant thereof, wherein the variant has at most two amino acid changes compared to the LCDR1;
v. LCDR2 set forth as SEQ ID NO. 107 or 131 or a variant thereof, wherein the variant has at most two amino acid changes compared to the LCDR2;
vi. LCDR3 set forth as SEQ ID NO. 108 or 132 or a variant thereof, wherein the variant has at most two amino acid changes compared to the LCDR3.

Further,
(i) the HCDR1, the HCDR2, and the HCDR3 is ected from a group set forth as: SEQ ID NO. 103, SEQ ID NO. 104, and SEQ ID NO. 105, respectively; or SEQ ID NO. 127, SEQ ID NO. 128, and SEQ ID NO. 129, respectively; and
(ii) each of the variants of the HCDR1, HCDR2, and HCDR3 has at most two amino acid substitutions, deletions or insertions compared to the corresponding HCDR1, HCDR2, or HCDR3 in (i);
(iii) LCDR1, LCDR2, and LCDR3 is selected from a group set forth as: SEQ ID NO. 106, SEQ ID NO. 107, and SEQ ID NO. 108, respectively; or SEQ ID NO. 130, SEQ ID NO. 131, and SEQ ID NO. 132, respectively; and
(iv) each of the variants of the LCDR1, LCDR2, and LCDR3 has at most two amino acid substitutions, deletions or insertions compared to the corresponding LCDR1, LCDR2, or LCDR3 in (iii).

In some embodiments, the antibody or antigen-binding fragment is specifically ACRO3-286L, ACRO3-347K. In some embodiments, the neutralizing antibody or the antigen-binding fragment capable of binding to the SARS-CoV-2 virus contains:
a) the heavy chain variable region having an amino acid sequence set forth as SEQ ID NO. 30 or SEQ ID NO. 46, or that is at least 70%, 80%, 90%, 95%, 99% or 100% similarity with SEQ ID NO. 30 or SEQ ID NO. 46; and
b) the light chain variable region having an amino acid sequence set forth as SEQ ID NO. 32 or SEQ ID NO. 48, or that is at least 70%, 80%, 90%, 95%, 99% or 100% similarity with SEQ ID NO. 32 or SEQ ID NO. 48.

The antibody or antigen-binding fragment further comprises a coupling moiety connected to a polypeptide, and the coupling moiety is selected from at least one of radionuclides, drugs, toxins, cytokines, enzymes, fluoresceins, carrier proteins, lipids, and biotin, wherein the polypeptide or the antibody is selectively linked to the coupling moiety by a linker, the linker is a peptide or a polypeptide.

Further, the antibody or antigen-binding fragment is selected from monoclonal antibodies, polyclonal antibodies, antiserum, chimeric antibodies, humanized antibodies, and human antibody; the antibody is selected from multispecific antibodies, single-chain Fv (scFv), single-chain antibodies, anti-idiotype (anti-Id) antibodies, diabodies, minibodies, nanobodies, single domain antibodies, Fab fragments, F(ab′) fragments, disulfide-linked bispecific Fv (sdFv) and intrabodies.

The second aspect of the present disclosure also provides a nucleic acid, wherein the nucleic acid encodes the antibody or antigen-binding fragment of the first aspect, preferably, the nucleic acid sequence combination includes SEQ ID NO. 41 and SEQ ID NO. 43, SEQ ID NO. 89 and SEQ ID NO. 91;

The third aspect of the present disclosure provides a recombinant vector comprising the nucleic acid of the second aspect, and an optional regulatory sequence; the recombinant vector may be a cloning vector or an expression vector, without limitation; further, the regulatory sequence can be selected from a leader sequence, a polyadenylation sequence, a propeptide sequence, a promoter, a signal sequence, a transcription terminator, or any combination thereof, without limitation.

The fourth aspect of the present disclosure provides a host cell comprising the nucleic acid of the second aspect or the recombinant vector of the third aspect; further, the host cell includes but not limited to yeast cells, Chinese hamster ovary cells, human embryonic kidney cells, other mammalian cells, and other cells suitable for the production of antibodies or antigen-binding fragments.

The fifth aspect of the present disclosure provides a pharmaceutical composition, comprising any one or more of the above-mentioned antibody or antigen-binding fragment, polynucleotide, recombinant vector, and host cell; preferably, the composition also comprises a pharmaceutically acceptable carrier or adjuvant.

The sixth aspect of the present disclosure provides a kit, comprising any one or more of the above-mentioned antibody or antigen-binding fragment, polynucleotide, recombinant vector, and host cell, wherein the kit is contained in a suitable container.

The seventh aspect of the present disclosure provides a method for preparing neutralizing antibodies or antigen-binding fragments capable of binding to the SARS-CoV-2 virus, wherein the method comprises expressing the third aspect vector in the host cell to produce the antibody; and recovering the antibody molecule from the cell culture.

The eighth aspect of the present disclosure provides a use of the antibody of the first aspect or an antigen-binding fragment in any of the following aspects: the treatment of COVID-19; the preparation of a pharmaceutical composition for the treatment of COVID-19; the prevention of COVID-19; the preparation of a COVID-19 vaccine; neutralizing antibody detection, screening, purification, and preparation; and f) the preparation of neutralizing antibody detection, screening, and purification kits.

The ninth aspect of the present disclosure provides a pharmaceutical composition, comprising the antibody or antigen-binding fragment against SARS-CoV-2 spike(S) protein of the first aspect, and a pharmaceutically optional pharmaceutical carrier.

The tenth aspect of the present disclosure provides a method for treating COVID-19, comprising administering to a subject an effective amount of the antibody or antigen-binding fragment of the first aspect, or the pharmaceutical composition of the eighth aspect.

### DETAILED DESCRIPTION

The present disclosure discloses an isolated antibody or an antigen-binding fragment, and those skilled in the art can refer to the content herein to realize its application. In particular, it should be pointed out that all similar substitutions and modifications are obvious to those skilled in the art, and they are all deemed to be included in the present disclosure. The preparation method and application of the present disclosure have been described through preferred embodiments, those skilled in the art can obviously make changes or appropriate changes and combinations to the preparation methods and applications herein without departing from the content, spirit, and scope of the application to realize and apply the technology of the application. Unless defined otherwise, all technical and scientific terms used herein have the same meaning as commonly understood by one of ordinary skill in the art to which this application belongs.

The following terms or definitions are provided only to help in understanding the present disclosure. These definitions should not be construed as having a scope less than that understood by those skilled in the art.

Unless otherwise defined hereinafter, the meanings of all technical and scientific terms used in the detailed description of the present disclosure are intended to be the same as those commonly understood by those skilled in the art. Although it is believed that the following terms are well understood by those skilled in the art, the following definitions are still set forth to better explain the application.

As shown in the present disclosure and claims, unless the context clearly prompts the exception, the terms “comprise,” “comprises,” and/or “comprising,” “include,” “includes,” and/or “including,” when used in the present disclosure, may be understood as implying inclusion of stated elements, but do not preclude the presence or addition of one or more other steps and elements thereof. The term “consisting of” is considered a preferred embodiment of the term “comprising”. If in the following a certain group is defined as comprising at least a certain number of embodiments, this is also to be understood as revealing a group which preferably consists only of these embodiments.

As shown in the present disclosure and claims, unless the context clearly prompts the exception, “a”, “an”, “one”, and/or “the” is not specifically intended to include singular form, and the plural form may be included.

The terms “approximately” and “about” in this present disclosure are employed to indicate a range of accuracy acceptable to those skilled in the art while still ensuring the desired technical effect of the mentioned features. In this context, the terms signify a range within ±10% of the indicated value, with a preference for ±5% to maintain the desired result.

The terms “or more”, “at least”, “more than”, etc., for example, “at least”, shall be understood as including but not limited to, at least 1, 2, 3, 4, 5, 6, 7, 8, 9, 10, 11, 12,13,14,15,16,17,18,19,20,21,22,23, 24,25,26,27,28,29,30,31,32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or 200, 300, 400, 500, 600, 700, 800, 900, 1000, 2000, 3000, 4000, 5000 of the stated value(s), or any higher numbers or fractions within this range.

Conversely, the term “not exceeding” includes every value less than the stated value. “Not more than 100 nucleotides” indicates, for example, 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 59, 60, 61, 58, 55, 56, 57, 51, 50, 52, 53, 54, 49, 48, 47, 46, 43, 44, 45, 42, 41, 38, 39, 40, 35, 34, 37, 36, 33, 32, 31, 28, 29, 30, 25, 26, 27, 24, 23, 22,19,20,21,18,17,16,13,14,15,12,11, 10,9, 8, 7, 6, 5, 4, 3, 2, 1 or 0 nucleotides, an/or any lower numbers or fractions within this range.

The terms “multiple”, “at least two”, “two or more”, “at least the second” should be understood as including but not limited to at least 2, 3, 4, 5, 6, 7, 8, 9, 10, 11, 12,13,14,15,16,17,18,19,20,21, 22,23,24,25,26,27,28,29,30,31,32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or 200, 300, 400, 500, 600, 700, 800, 900, 1000, 2000, 3000, 4000, 5000 or more, and/or any higher numbers or fractions within this range.

The following are definitions of some terms used in the present disclosure.

As used in the present disclosure, the term “antibody” refers to a polypeptide of the immunoglobulin family capable of non-covalent, reversible, and specific binding to a corresponding antigen. For example, a naturally occurring IgG antibody is a tetramer comprising at least two heavy (H) chains and two light (L) chains interconnected by disulfide bonds. Each heavy chain comprises a heavy chain variable region (abbreviated herein as VH) and a heavy chain constant region.

The heavy chain constant region consists of three domains CH1, CH2, and CH3. Each light chain comprises a light chain variable region (abbreviated herein as VL) and a light chain constant region. The light chain constant region contains a domain CL. The VH and VL regions can be further subdivided into hypervariable regions known as complementarity-determining regions (CDRs). The hypervariable regions are interspersed with more conserved regions known as framework regions (FR). Each VH and VL consists of three CDRs and four FRs arranged from amino-terminus to carboxy-terminus in the following order: FR1, CDR1, FR2, CDR2, FR3, CDR3, and FR4. The variable regions of the heavy and light chains contain binding domains that interact with antigen. The constant regions of the antibodies can mediate the binding of the immunoglobulin to host tissues or factors, including various cells of the immune system (e.g., effector cells) as well as the first component (Clq) of the classical complement system. The “antibody” includes, but be not limited to, monoclonal antibodies, human antibodies, humanized antibodies, camelid antibodies, chimeric antibodies, and anti-idiotypic (anti-Id) antibodies (including, for example, anti-Id antibodies directed against antibodies of the disclosure). Antibodies can be of any isotype/class (e.g., IgG, IgE, IgM, IgD, IgA, and IgY) or subclass (e.g., IgG1, IgG2, IgG3, IgG4, IgA1, and IgA2).

Antibodies comprise globular regions known as “domains” within heavy or light chain polypeptides. These domains may comprise peptide loops, usually 3 to 4 loops, which are stabilized, for example, by β sheets and/or intrachain disulfide bonds. The nomenclature of “constant” or “variable” domains is based on the level of sequence variation observed within domains of different classes. In the case of “constant” domains, there is relatively little sequence variation, whereas “variable” domains display significant variation among different classes. The terms “antibody domains” and “antibody regions” can be used interchangeably in the present disclosure.

Antibodies can be divided into five classes: IgA, IgD, IgE, IgG, and IgM, based on the amino acid sequence of the constant region of the antibody's heavy chain, and several isotypes within these classes can be further divided into subclasses, such as IgG1, IgG2, IgG3, IgA1, and IgA2. The heavy chain constant domains corresponding to the different classes of immunoglobulins are called a, 5, E, y, and p, respectively. K and A can be divided according to the difference of antibody's light chain constant region (CL). Within full-length light and heavy chains, typically the variable and constant regions are joined by a “J” region of about 12 or more amino acids, with the heavy chain also including a “D” region of about 10 more amino acids.

The term “monoclonal antibody” refers to a type of antibody which binds only to the same epitope (the part of an antigen that is recognized by the antibody), regardless of the method by which it is prepared. Monoclonal antibodies or immunologically active fragments can be produced by hybridoma technology, recombinant technology, phage display technology, synthetic technology, etc., or other production techniques known in the art. The methods involved in the preparation of monoclonal antibodies in this present disclosure include in vitro culture of hybridoma cells or DNA recombinant technology. The monoclonal antibodies are highly specific, targeting a same antigenic site. Each monoclonal antibody is directed against a single determinant on the antigen.

The term “antigen” refers to an entity (e.g., protein entity or peptide) to which an immunoglobulin or antibody (or antigen-binding fragment) specifically binds.

The term “fragment” may refer to a portion or portion of an antibody or antibody chain comprising fewer amino acid residues than an intact or complete antibody or antibody chain, wherein the portion preferably retains at least one, preferably most, or all of the functions normally associated with this portion when present in an intact antibody. The fragments can be obtained by chemical or enzymatic treatment of intact or complete antibodies or antibody chains. The fragments can also be obtained by recombination.

The term “variable” refers to some portion of the variable region of an antibody that exhibit sequence differences, which contributes to the binding and specificity of each particular antibody for its particular antigen. These variable regions, however, do not possess an even distribution of variability. Instead, they typically concentrate within three segments known as complementarity determining regions (CDRs) or hypervariable regions found within both the light and heavy chain variable regions. The more conserved part of the variable region is referred to as the framework region (FR). The variable domains of the native heavy and light chains each contain four FR regions in a roughly beta-sheet configuration connected by three CDRs that form connecting loops, in some cases forming partial p sheet structures. The close proximity of CDRs within each chain is facilitated by the FR regions, and together with the CDRs of the opposite chain, these regions constitute the antigen-binding site of the antibody (see Kabat et al., NIH Publ. No. 91-3242, Vol. I, pp. 647-669 (1991). The constant regions are not directly involved in the binding of antibodies to antigens, but they exhibit different effector functions, such as participation in antibody-dependent cytotoxicity.

The term “complementarity-determining domains” or “complementarity-determining regions” (“CDRs”) refer interchangeably to the hypervariable regions of VL and VH. CDR is the target protein binding site of the antibody chain carrying the specificity of this target protein. Three CDRs (CDRs1-3, numbered sequentially from the N-terminus) are present in each VL or VH, accounting for approximately 15-20% of the variable domain in total. CDR can be referred to by its regions and order. For example, “VHCDR1” or “HCDR1” both refer to the first CDR of the heavy chain variable region. CDRs are structurally complementary to the epitope of the target protein and are thus directly responsible for binding specificity. The remaining stretches of VL or VH (so-called framework regions) exhibit less variation in amino acid sequence (Kuby, Immunology, 4th Edition, Chapter 4 W. H. Freeman & Co., New York, 2000).

In a given light chain variable region or heavy chain variable region amino acid sequence, the precise amino acid sequence boundaries of each CDR can be determined using any one or combination of well-known antibody CDR numbering systems (or schemes), for example, the Chothia system based on the three-dimensional structure of the antibody and the topology of the CDR loops (Chothia et al. (1989) Nature 342:877-883; AI-Lazikani et al, “Standard conformations for the canonical structures of immunoglobulins”, Journal of Molecular Biology, 273, 927-948(1997)), the Kabat system based on antibody sequence variability (Kabat et al., Sequences of Proteins of Immunological Interest, 4th ed., U.S. Department of Health and Human Services, National Institutes of Health (1987)), AbM (University of Bath), Contact (University College London), International ImMunoGeneTics database (IMGT), and the North CDR definition based on affinity propagation clustering using a large number of crystal structures.

However, it should be noted that the boundaries of the CDRs of the variable regions of the same antibody obtained based on different numbering systems may vary. That is, the CDR sequences of the same antibody variable region defined under different numbering systems are different. For example, the residue ranges defined by different numbering systems for CDR regions are shown in the table below.

CDR residue ranges defined by different numbering systems

| | Kabat CDR | AbM | Chothia | Contact | IMGT |
|---|---|---|---|---|---|
| L1 | L24-L34 | L24-L34 | L24-L34 | L30-L36 | L27-L32 |
| L2 | L50-L56 | L50-L56 | L50-L56 | L46-L55 | L50-L52 |
| L3 | L89-L97 | L89-L97 | L89-L97 | L89-L96 | L89-L96 |
| H1 | H31-H35b | H26-H35b | H26-H32 . . . 34 | H30-H35b | H26-H35b |
| | Kabat | | | | |
| | numbering | | | | |
| H1 | H31-H35b | H26-H35 | H26-H32 | H30-H35 | H26-H35 |
| | Chothia | | | | |
| | numbering | | | | |
| H2 | H50-H65 | H50-H58 | H52-H56 | H47-H58 | H51-H57 |
| H3 | H95-H102 | H95-H102 | H95-H102 | H93-H101 | H93-H102 |

Thus, where reference is made to defining an antibody with a particular CDR sequence as defined in the present disclosure, the scope of said antibody also encompasses antibodies whose variable region sequences comprise said particular CDR sequences. However, due to the application of different schemes (such as different numbering system rules or combinations), the claimed CDR boundaries may be different from the specific CDR boundaries defined in this present disclosure.

The CDRs of the antibodies of the present disclosure can be manually evaluated to determine the boundaries according to any scheme in the art or a combination thereof. As shown in the present disclosure and claims, unless the context clearly prompts the exception, the term “CDR” or “CDR sequence” encompasses a CDR sequence determined in any of the above-mentioned ways.

The antibodies may include, for example, monoclonal antibodies, recombinant antibodies, single specific antibody, dual specific antibody (including bispecific antibody), human antibody, engineered antibody, humanized antibody, chimeric antibody, immune globulin, synthetic antibody, four polymers antibodies containing two heavy chain and light chain molecules, antibody light chain monomer, antibody heavy chain monomer, antibody light chain dimers, antibody heavy chain dimers, light chain—heavy chain pairs, intracellular antibodies, antibody fusion (sometimes referred to as the “antibody conjugate”), conjugated antibodies, single domain antibody, univalent antibody, single chain or single chain Fv antibodies (scFv), camel-derived antibody, Fab fragments, F (ab′) 2 fragments, disulfide bond-connected Fv (sdFv), special type (Id) resistance antibodies (including, for example, fight, fight Id antibody), micro antibodies, domain antibodies, synthetic antibody (sometimes called “antibody analog”) and the antigen-binding fragments of the above antibodies.

The term “antigen-binding fragment” refers to one or more portions of an antibody that retain the ability to specifically interact (e.g., by binding, steric hindrance, stabilization/destabilization, spatial distribution) with an epitope of an antigen. Examples of binding fragments include, but are not limited to, single chain Fv (scFv), disulfide-linked Fv (sdFv), Fab fragment, F(ab′) fragment(i.e. a monovalent fragment consisting of VL, VH, CL and CH1 domains); F (ab) 2 fragments (including in the hinge region by disulfide bridge connecting two Fab fragments of bivalent fragments); the Fd fragments consisting of VH and CH1 domains; Fv fragments consisting of the VL and VH domains of a single arm of an antibody; dAb fragments composed of VH domains (Ward et al., Nature 341:544-546, 1989); and isolated complementarity determining regions (CDRs) or other epitope-binding fragments of antibodies. Furthermore, although the two domains VL and VH of the Fv fragment are encoded by separate genes, the two domains can be linked using recombinant methods through a synthetic linker that enables the two domains to become a single protein chain in which the pair of VL and VH regions is used to form a monovalent molecule (referred to as a single-chain Fv (“scFv”).; See, e.g., Bird et al., Science 242:423-426, 1988; and Huston et al., Proceedings of the National Academy of Sciences USA 85:5879-5883, 1988). Such single chain antibodies are also intended to be encompassed within the term “antigen-binding fragment”. These antigen-binding fragments are obtained using routine techniques known to those skilled in the art, and the fragments are screened for utility in the same manner as whole antibodies.

The antigen-binding fragments can also be incorporated into single domain antibodies, macrobodies, minibodies, nanobodies, intrabodies, diabodies, triabodies, tetrabodies, v-NAR, and bis-scFv (see, e.g., Hollinger and Hudson, Nature Biotechnology 23:1126-1136, 2005). Antigen-binding fragments can be based on polypeptides such as fibronectin type III (Fn3) grafted into scaffolds (see U.S. Pat. No. 6,703,199 which describes fibronectin polypeptide mAbs). Antigen-binding fragments can be incorporated into single-chain molecules comprising a pair of tandem Fv segments (VH-CH1-VH-CH1) that together form a pair of antigen-binding regions with complementary light chain polypeptides (Zapata et al., Protein Eng. 8:1057-1062, 1995; and (U.S. Pat. No. 5,641,870)).

The term “chimeric antibody” refers to a part of the heavy chain and/or light chain (generally the variable region) derived from a specific species or belonging to a specific antibody class or subclass of the corresponding sequence identical or homologous, while the rest of the chain (generally referred to as the constant region) derived from another species of antibody or belonging to another antibody class or subclass of antibodies, and the corresponding sequences in fragments of these antibodies are identical or homologous, as long as they exhibit the desired biological activity. The chimeric antibody involved in the present disclosure, such as the heavy chain/light chain variable region from a murine antibody, is grafted to the heavy chain/light chain constant region of a human antibody through antibody engineering technology, which exhibits similar biological activities.

The term “humanized antibody” refers to a chimeric antibody comprising amino acid residues from non-human HVRs and amino acid residues from human FRs. In some embodiments, a humanized antibody comprises substantially all of at least one, usually two, variable domains, wherein all or substantially all of the HVRs (e.g., CDRs) correspond to the sequence of a non-human antibody and all or substantially all of the FRs correspond to the sequence of a human antibody.

The term “affinity” refers to the strength of the interaction between an antibody and an antigen at a single antigenic site. Within each antigenic site, the variable regions of the antibody “arm” interact with the antigen at many sites through weak non-covalent forces. The more interactions, the stronger the affinity.

As shown in the present disclosure and claims, the term “competes” when used in the context of antigen binding proteins (e.g., neutralizing antigen binding proteins or neutralizing antibodies) that compete for the same epitope, means competition between antigen binding proteins as determined by an assay in which the antigen binding protein (e.g., antibody or immunologically functional fragment) to be tested prevents or inhibits (e.g., reduces) specific binding of a reference antigen binding protein (e.g., ligand or reference antibody) to a common antigen.

As shown in the present disclosure, the term “variant” refers to a heavy chain variable region or a light chain variable region that has been modified by at least one, such as 1, 2, or 3 amino acid substitutions, deletions, or additions, wherein the modified antigen binding protein comprising the heavy chain or light chain variant substantially retains the biological characteristics of the pre-modified antigen binding protein.

In some embodiments, the antigen binding protein comprising a variant heavy chain variable region or light chain variable region sequence retains 70%, 80%, 90%, 100% of the biological characteristics of the modified pre-antigen binding protein. It is understood that each heavy or light chain variable region can be modified alone or in combination with another heavy or light chain variable region. Antigen binding proteins of the present disclosure comprise heavy chain variable region amino acid sequences that are 70%, 75%, 80%, 85%, 90%, 91%, 92%, 93%, 94%, 95%, 96%, 97%, 98%, or 99% homologous to the heavy chain variable region amino acid sequences of the modified pre-antigen binding protein. Antigen binding proteins of the present disclosure comprise light chain variable region amino acid sequences that are 70%, 75%, 80%, 85%, 90%, 91%, 92%, 93%, 94%, 95%, 96%, 97%, 98%, or 99% homologous to the light chain variable region amino acid sequences of the modified pre-antigen binding protein.

The percent homology may be over the entire heavy chain variable region and/or the entire light chain variable region, or the percent homology may be limited to the framework regions, while the sequences corresponding to the CDRs are 100% identical to the CDRs disclosed herein within the heavy chain variable region and/or the light chain variable region. As shown in the present disclosure, the term “CDR variant” refers to a CDR that has been modified by at least one, for example, 1, 2, or 3 amino acid substitutions, deletions, or additions, wherein the modified antigen binding protein comprising the CDR variant substantially retains the biological characteristics of the pre-modified antigen binding protein. In some embodiments, the antigen binding protein containing the variant CDR retains 60%, 70%, 80%, 90%, and 100% of the biological characteristics of the antigen binding protein before modification. It is understood that each CDR that can be modified alone or in combination with another CDR. In an embodiment, the modification is a substitution, especially a conservative substitution.

The term “vector” in the present disclosure refers to a nucleic acid molecule capable of amplifying by transformation of another nucleic acid to which it has been linked. The term includes vectors that are self-replicating nucleic acid structures as well as vectors that integrate into the genome of a host cell into which they have been introduced. Some vectors are capable of directing the expression of nucleic acids to which they are operably linked. The vectors may be referred to as “expression vectors” in the present disclosure.

The term “host cell” refers to a cell that has been infused with external nucleic acid, along with its offspring, and possesses the ability to manifest the introduced nucleic acid within the cell or cell membrane, or release it outside the cell.

The term “subject” comprises human and non-human animals. Non-human animals comprises all vertebrates such as mammals and non-mammals such as non-human primates, sheep, dogs, cows, chickens, amphibians, and reptiles As shown in the present disclosure and claims, unless the context clearly prompts the exception, the term “patient” or “subjects” can be used interchangeably.

The term “neutralizing” or “neutralization” refers to the inhibition of viral infection of a host cell, as demonstrated by the absence of viral gene expression. Without being bound by any one theory, the mechanism of neutralization by specific antibodies may involve blocking the interaction of viral capsid proteins with cell surface receptors or disrupting any stage of the entry and trafficking process prior to the delivery of the viral genome to the nucleus of the host cell.

As shown in the present disclosure, on one hand, the term “treatment” of any disease or disorder may refer to alleviating the disease or disorder (i.e., slowing or arresting, or reducing the development of at least one of the disease or its clinical symptoms). “Treatment”, on the other hand, refers to the alleviation or improvement of at least one physical parameter, including those physical parameters that the patient may not be able to discern. In another aspect, “treating” may refer to modulating a disease or disorder physically (e.g., stabilizing discernible symptoms), physiologically (e.g., stabilizing a physical parameter), or both.

The term “kit” may refer to a combination of reagents and other materials that facilitate the analysis of a sample. In some embodiments, the immunoassay kits in the present disclosure comprise a suitable antigen, a binding agent comprising a detectable moiety, and a detection reagent. Systems for amplifying the signal generated by the detectable moiety may also or may not be included in the kit.

Additionally, in some other embodiments, kits include, but are not limited to, components such as devices for sample collection, sample tubes, racks, trays, racks, plates, plates, kit user's instructions, solutions, or other chemical reagents, and samples for normalization, normalization, and/or control samples.

The term “pharmaceutically acceptable” means that the carrier, diluent, excipient and/or salt thereof is chemically and/or physically compatible with the other ingredients of the formulation and physiologically compatible with the recipient.

The term “pharmaceutically acceptable carrier and/or excipient” refers to a carrier and/or excipient pharmacologically and/or physiologically compatible with the subject and the active agent, which are well known in the art (see, e.g., Remington's Pharmaceutical Sciences. Edited by Gennaro AR, 19th ed. Pennsylvania: Mack Publishing Company, 1995), and include, but are not limited to, pH adjusting agents, surfactants, adjuvants and ionic strength enhancers. For example, PH adjusting agents include, but are not limited to, phosphate buffer; Surfactants include but are not limited to cationic, anionic or nonionic surfactants such as Tween-80; Ionic strength enhancers include, but are not limited to, sodium chloride.

The novel coronavirus is targeted by the antibody of this disclosure.

Coronaviruses (including SARS-CoV and the newly discovered 2019-nCoV) are spherical single-stranded positive-sense RNA viruses characterized by a spike protein protruding from the surface of the virion (Barcena, M. et al., Cryo-electron tomography of mouse hepatitis virus: Insights into the structure of the coronavirus. Proc. Natl. Acad. Sci. USA 2009, 106, 582-587). The spherical shape of the virus particles and the spikes make the coronavirus look like a crown under the electron microscope, so it was named coronavirus.

Coronavirus is an enveloped virus (the envelope is derived from the lipid bilayer of the host cell membrane), with a viral structure mainly formed by viral structural proteins (such as Spike, S, Membrane, M, Envelope, E, and Nucleocapsid, N), in which the S, M, and E proteins are all embedded in the viral envelope, and the N protein interacts with the viral RNA and is located at the core of the virus particle to form a nucleocapsid (Fehr, A. R. et al., Coronaviruses: An overview of their replication and pathogenesis. Methods Mol. Biol. 2015, 1282, 1-23). The S protein is a highly glycosylated protein that forms homotrimeric spikes on the surface of virus particles and mediates viral entry into host cells.

2019-nCoV is a single-stranded positive-strand RNA virus with a membrane structure and a size of 80-120 nm. The genome length is about 29.9 kb. The homology between this virus and the genome sequence of SARS-CoV belonging to the genus Betacoronavirus of the Coronaviridae family is 80%. The open reading frame (ORF) ORF1 a and ORF1 b of the viral genome account for ⅔ of the genome, and express hydrolases and enzymes related to replication and transcription, such as cysteine protease (PLpro) and serine protease (3CLpro), RNA-dependent RNA polymerase (RdRp) and helicase (Hel). The latter ⅓ region of the genome is mainly responsible for coding structural proteins, including spike protein (S), envelope protein (E), membrane protein (M), nucleocapsid protein (N) and other main structural proteins, in which the N protein wraps the viral genome to form a nucleoprotein complex, the E protein, and the M protein are mainly involved in the assembly process of the virus, and the S protein mainly mediates the invasion of the virus and determines the host specificity of the virus by binding to the host cell receptor.

After sequence comparison, it was found that the S protein of 2019-nCoV virus and SARS-CoV virus has a similarity of 75%. It is reported that the amino acid residues at positions 442, 472, 479, 487, and 491 of the complex interface between S protein and ACE2 receptor (mainly distributed in respiratory epithelial cells, lungs, heart, kidneys, and digestive tracts in humans) in multiple SARS-CoV coronavirus isolates are highly conserved. Compared with the S protein of SARS-CoV, at the 5 sites, the 2019-nCoV S protein only has the same 491st amino acid, and the other 4 amino acids have mutations (Xu X et al., Sci China Life Sci., March 2020; 63(3):457-460). Nevertheless, through protein 3D structure simulation prediction, it was found that although the four key amino acids that bind the 2019-nCoV S protein to the ACE2 receptor have all been replaced, the three-dimensional structure of the receptor binding domain (RBD) in the 2019-nCoV S protein is almost unchanged compared to the SARS-CoV S protein, so the 2019-nCoV S protein still has a high affinity with human ACE2 EM structure of the 2019-nCoV Spike disclosed in (the Prefusion Conformation, Science, Feb. 19, 2020, published online, pii: eabb2507.doi:10.1 126/science.abb2507) and (Xiaolong Tian et al., Potent binding of 2019 novel coronavirus spike protein by a SARS coronavirus-specific human monoclonal antibody, Emerging Microbes & Infections, 2020, 9:1, p382-385, DOI: 10.1080/22221751.2020.1729069). Detected by Fortebio, the affinity (KD) of the S protein of 2019-nCoV binding to human ACE2 is about 15 nM, The binding affinity of the S protein of SARS-CoV to human ACE2 is comparable. It can be seen that ACE2 is also the receptor protein for 2019-nCoV to infect the human body and enter the cell interior. It is expected that a high-affinity neutralizing antibody directed against the coronavirus S protein and blocking its binding to the ACE2 receptor can effectively prevent and treat coronavirus (e.g., 2019-n CoV) infection.

As the global epidemic continues to spread, the new coronavirus is also constantly mutating. All mutant strains, such as the latest Delta mutant, Omicron mutant, etc., belong to the category of new coronaviruses in this disclosure.

1. The antibody or antigen-binding fragment against SARS-CoV-2 spike(S) protein.

The terms “antibody against SARS-CoV-2 spike(S) protein”, “antibody against the coronavirus S protein”, “anti-S protein antibody”, “coronavirus S protein antibody”, “S protein antibody” or “S protein-binding antibody” are used interchangeably in the present disclosure to refer to an antibody or antigen-binding fragment of the present disclosure that is capable of binding to the coronavirus S protein (e.g., 2019-n CoV S protein, SARS-CoV S protein) with sufficient affinity, whereby the antibody can be used to target the coronavirus S protein for diagnosis, prevention and/or treatment.

The antibodies and antigen-binding fragments of the present disclosure specifically bind to the coronavirus S protein with high affinity. In some embodiments, the antibodies of the present disclosure are blocking or neutralizing antibodies. In some embodiments, the blocking antibody or neutralizing antibody can be used to prevent coronavirus infection and/or treat a coronavirus-infected individual.

In some embodiments, according to the different analysis methods for CDR in the above definition, the coronavirus S protein antibody or antigen-binding fragment of the present disclosure includes:

three complementarity determining regions (HCDRs) of a heavy chain variable region or one or more variants thereof, the heavy chain variable region set forth as SEQ ID NO. 30 or SEQ ID NO. 46, each of the one or more variants having at most two amino acid changes compared to the corresponding CDR; and three complementarity determining regions (LCDRs) of a light chain variable region or one or more variants thereof, the light chain variable region set forth as SEQ ID NO. 32 or SEQ ID NO. 48, each of the one or more variants having at most two amino acid changes compared to the corresponding CDR.

In some embodiments, the HCDRs and LCDRs are numbered by a Kabat numbering scheme, and the antibody or the antigen-binding fragments includes:
i. HCDR1 set forth as SEQ ID NO. 103 or 127 or a variant thereof, wherein, the variant has at most two amino acid changes compared to the HCDR1;
ii. HCDR2 set forth as SEQ ID NO. 104 or 128 or a variant thereof, wherein the variant has at most two amino acid changes compared to the HCDR2;
iii. HCDR3 set forth as SEQ ID NO. 105 or 129 or a variant thereof, wherein the variant has at most two amino acid changes compared to the HCDR3;
iv. LCDR1 set forth as SEQ ID NO. 106 or 130 or a variant thereof, wherein the variant has at most two amino acid changes compared to the LCDR1;
v. LCDR2 set forth as SEQ ID NO. 107 or 131 or a variant thereof, wherein the variant has at most two amino acid changes compared to the LCDR2;
vi. LCDR3 set forth as SEQ ID NO. 108 or 132 or a variant thereof, wherein the variant has at most two amino acid changes compared to the LCDR3.

In some embodiments,
(i) the HCDR1, the HCDR2, and the HCDR3 are selected from a group set forth as: SEQ ID NO. 103, SEQ ID NO. 104, and SEQ ID NO. 105, respectively; or SEQ ID NO. 127, SEQ ID NO. 128, and SEQ ID NO. 129, respectively; and (ii) each of the variants of the HCDR1, HCDR2, and HCDR3 has at most two amino acid substitutions, deletions or insertions compared to the corresponding HCDR1, HCDR2, or HCDR3 in (i);
(iii) LCDR1, LCDR2, and LCDR3 is selected from a group set forth as: SEQ ID NO. 106, SEQ ID NO. 107, and SEQ ID NO. 108, respectively; or SEQ ID NO. 130, SEQ ID NO. 131, and SEQ ID NO. 132, respectively; and
(iv) each of the variants of the LCDR1, LCDR2, and LCDR3 has at most two amino acid substitutions, deletions or insertions compared to the corresponding LCDR1, LCDR2, or LCDR3 in (iii).

In some specific embodiments, the antibody or antigen-binding fragment is ACRO3-286L, ACRO3-347K. In some specific embodiments, the antibody or the antigen-binding fragment includes:
a) the heavy chain variable region having an amino acid sequence set forth as SEQ ID NO. 30 or SEQ ID NO. 46, or that is at least 70%, 80%, 90%, 95%, 99% or 100% similarity with SEQ ID NO. 30 or SEQ ID NO. 46; and
b) the light chain variable region having an amino acid sequence set forth as SEQ ID NO. 32 or SEQ ID NO. 48, or that is at least 70%, 80%, 90%, 95%, 99% or 100% similarity with SEQ ID NO. 32 or SEQ ID NO. 48.

In some embodiments, the amino acid changes do not occur in the CDR regions.

In some more specific embodiments, some specific antibody sequences of this present disclosure are shown in the following table (the specific VH and VL sequences of neutralizing antibodies or antigen-binding fragments thereof binding to SARS-CoV-2 virus).

| Anti- | | SEQ | |
|---|---|---|---|
| bodies | | NO. | Antibody Sequences |
| ACRO3- | VH | 30 | QVQLVQSGAEVKKPGASVKVSCKASGYTFSSYYIHWVRQAPGQ |
| 286L | | | GPEWMAIINPGDGGASYAQKFQGRVTLTRDTSTSTLYMELSSL |
| | | | RSEDTAVYYCARAEGSSWLGWFDPWGQGTLVTVSS |
| | VL | 32 | QSVLTQPPSASGTPGQRVTISCSGSSSNIGSNYVYWYQQLPGT |
| | | | APKLLIYRNNQRPSGVPDRFSGSRSGTSASLAISGLRSEDEAD |
| | | | YYCAAWDDGLSGSGWVFGGGTKLTVL |
| ACRO3- | VH | 46 | QMQLVQSGPEVKKPGTSVKVSCKASGFTFTDVSSLQWVRQARG |
| 347K | | | QRLEWIGWTVVGTGNTNYAPRFQERVTITTDKSTSTAYMELSS |
| | | | LRSEDTAVYYCAAPFCSETSCSDGFDLWGQGTKVTVSS |
| | VL | 48 | EIVLTQSPGTLSLSPGDRATLSCRASQSVRISYLAWYQQKPGQ |
| | | | APRLLISGSSSRATGIPDRFSASGSGTDFALTISRLEPEDFAV |
| | | | YYCQQYDNSPWTFGQGTKVEVK |
| ACRO2- | VH | 2 | EVQLVESGGGVIQPGGSLRLSCAASGFTVSANYMSWVRQAPGK |
| 001K | | | GLEWVSVIYSGGSTNDGDTYYAASVKGRFTISRDNSKNTLYLQ |
| | | | MNSLRAEDTAVYYCARVVSDAFDIWGQGAMVTVSS |
| | VL | 4 | DIQMTQSPSTLSASVGDRVTITCRASQSISSWLAWYQQKPGKA |
| | | | PKLLIYKASILESGVPSRFSGSGSGTEFTLTISSLQPDDFATY |
| | | | YCQHYNSDSYTFGQGTKLEIK |
| ACRO2- | VH | 6 | EVQLLESGGGLVQPGGSLRLSCAASGFTFSSFAMTWVRQAPGK |
| 021L | | | GLECVSVISGGAGSTYYADSVKGRFTLSRDNSKNTLYLQMNSL |
| | | | RAEDTAVYYCAKGERPDYGDYFDYWGQGTLVTVSS |
| | VL | 8 | SYVLTQPPSVSVAPGKTARITCGGNNIGSKSVHWYQQRPGQAP |
| | | | VLVMYYDSDRPSGIPDRFSGSNSGNTATLTISRVEAGDEADYY |
| | | | CQVWDGSSDHHYVFGTGTKLTVL |
| ACRO2- | VH | 10 | EVQLVESGGGLVQPGGSLRLSCAASGFSVSSNYMSWVRQAPGK |
| 028K | | | GLEWVSVIYSGGSTYYADSVKGRFTISRHNSKNTLYLQMNSLR |
| | | | AEDTAVYYCARVVTDAFDIWGQGTMVTVSS |
| | VL | 12 | DIQMTQSPSTLSASVGDRVTITCRASQSISSWLAWYQQTPGKA |
| | | | PKLLIYKASSLESGVPSRFSGSGSGTEFTLTISSLQPDDFAIY |
| | | | YCQQYNSDSYTFGQGTKLEIK |
| ACRO3- | VH | 14 | QLQLQESGPGLVKPSETLSLTCTVSGGSISSSRYYWGWIRQPP |
| 168L | | | GKGLEWIGSLYYSGGTYFNPSLKSRVTISVDTSKNQFSLKLTS |
| | | | VTAADTAVYYCARDHYGDYALVYWGQGTLVTVSS |
| | VL | 16 | QSVLTQPPSVSGAPGQRVTISCTGSSSNIGAGYDVHWYQQLPG |
| | | | TAPKLLIYGHNNRPSGVPDRFSGSQSGTSASLAITGLQAEDEA |
| | | | DYYCQSYDTSLSALYVFGTGTKVTVL |
| ACRO3- | VH | 18 | QLQLQESGPGLVKPSETLSLTCTVSGGSISSSRYYWGWIRQPP |
| 169L | | | GKGLEWIGSLYYSGGTYYNPSLKSRVTISVDTSKNQFSLKLTS |
| | | | VTAADTAVYYCARDHYGDYALVYWGQGTPVTVSS |
| | VL | 20 | QSVLTQPPSVSGAPGQRVTISCTGSSSNVGAGYDVHWYQQLPG |
| | | | TAPKLLIYGNNNRPSGVPDRFSGSQSGTSASLAITGLQTEDEA |
| | | | DYYCQSYDTSLSALYVFGTGTKVTVL |
| ACRO3- | VH | 22 | QVQLVQSGSELKKPGASVKVSCKASGYSFTSYAMNWVRQAPGQ |
| 207L | | | GLEWMGWINTNTGNPTYAQGFTGRFVFSLDTSVNTAYLQISGL |
| | | | KAEDTAVYYCARETSWDGYIYWGQGTLVTVSS |
| | VL | 24 | SYEVTQPPSVSVSPGQTARITCSGDTLSKQYTFWYQQKTGQAP |
| | | | VLVIYKDSERPSGIPERFSGSSSGTTVTLTISGVQAEDEADYY |
| | | | CQSADNSGTSWVFGGGTKLTVL |
| ACRO3- | VH | 26 | QVQLVQSGAEVKKPGASVKVSCKVSGYTLIELSMHWVRQAPGK |
| 283L | | | GLEWMGGFDPEDAETIYAQKFQGRVTMTEDTSTDTAYMELSRL |
| | | | RSEDTAVYYCAIGPAIPMAATGWFDPWGQGTLVTVSS |
| | VL | 28 | QSALTQPASVSGSPGQSITISCTGTSSDVGGYNYVSWYQQHPG |
| | | | KAPKLMIYEVSNRPSGVSNRFSGSKSGNTASLTISGLQAEDEA |
| | | | DYYCSSYTSSSTYVFGTGTKVTVL |
| ACRO3- | VH | 34 | QVQLVESGGGVVQPGRSLRLSCSASGFIFSNYAMHWVRQAPGK |
| 307K | | | GLEWVAVIWDDGSNKHYADSVKGRFTISRDNSKNTLYLQMNSL |
| | | | RAEDTAVYYCARDQGELVTYFDYWGQGTLVIVSS |
| | VL | 36 | DIQMTQSPSSLSASVGDRVTITCRASQTISSYLNWYQQKPGKA |
| | | | PKLLIYTTSSLQSGVPSRFSGSGSGTDFTLTISSLQPEDFATY |
| | | | YCQQSYSTPSWTFGQGTKVEIK |
| ACRO3- | VH | 38 | EVQLVESGGGLVQPGGSLRLSCGASGFSFSNYDIHWVRQAIGT |
| 309K | | | GLEWVSCVGTYGDTYYSDSVKGRFTISRENAKNSLYLQMNSLR |
| | | | LGDTAVYFCARGSVDPTTNWFFDLWGRGTLVTVSS |
| | VL | 40 | DIQMTQSPSSLSASVGDRVTITCRASQGIRNFLAWYQQKPGKA |
| | | | PKLLLSAASRLESGVPSRFSGSGSGTDYTLTISSLQPEDFATY |
| | | | YCQQYYSSPPITFAQGTRLEIK |
| ACRO3- | VH | 42 | EVQLVESGGGLVQPGGSLRLSCAASGFTFSSYSMNWVRQAPGK |
| 341L | | | GLEWVSYISSSNITIYYADSVKGRFTISRDNAKNSLYMQVNSL |
| | | | RDEDTAVYYCARVLRYYDYVWGSYQRELYGMDVWGQGTTVTVS |
| | | | S |
| | VL | 44 | QSALTQPASVSGSPGQSITISCTGTSSDVGGYDYVSWYQQHPG |
| | | | KAPKLMIYDVSNRPSGVSNRFSGSKSGNTASLTISGLQAEDEA |
| | | | DYYCSSFTSSSTLPFVIGTGTKVTVL |
| ACRO3- | VH | 50 | QVQLVQSGAEVKKPGASVKVSCKASGYTFTNYGISWVRQAPGQ |
| 410K | | | GLEWMGWISSYNGNTHYARKLQGRVTMTTDTSTSTVYMELRSL |
| | | | RSDDTAVYYCARENFNYGDYIIYFDPWGQGTLVTVSS |
| | VL | 52 | EIVLTQSPDFQSVTPKEKVTITCRASESIGSSLHWYQQKPDQS |
| | | | PKLLIKHASQSFSGVPSRFSGSGSGTDFTLTINGLEAEDAATY |
| | | | YCHQSSSLPYTFGQGTKLEIK |
| ACRO3- | VH | 54 | QLQLQESGPGLVKPSETLSLTCTVSGGSISSGNYYWGWIRQPP |
| 411L | | | GKGLEWIGNIYYSGSTYYNPSLKSRVTLSVDTPKNQFSLKLSS |
| | | | VTAADTAVYYCARHVFQLLFWDNWFDPWGQGTLVTVSS |
| | VL | 56 | QSVLTQPPSASGTPGQRVTISCSGSSSNIGSNSVNWYQQIPGP |
| | | | APKLLIHSNNQRPSGVPDRFSGSKSGTSASLTISGIQSEDEAD |
| | | | YYCAAWDDSRNGVVFGGGTKLTVL |
| ACRO3- | VH | 58 | QVQLVESGGGVVQPGKSLRVSCAASGFPFSRFALHWVRQAPGK |
| 414K | | | GLEWVALISFDGTNKFYADSVKGRFTISRDNSKNIMYLDMNSL |
| | | | GDEDTAVYYCAKVVSGWHLVTYGLDVWGQGTTVTVSS |
| | VL | 60 | DIVMTQSPLSLSVTPGEPASISCRSSQSLLHTNGYDYLDWYLQ |
| | | | KPGQSPQLLMYLGSNRGSGVPVRFSASGTGTDFTLKISRVEAE |
| | | | DVGVYYCMQALQTPYTFGQGTKLEIK |

In some embodiments, the antibody or antigen-binding fragment of the present disclosure further comprises an Fc region from an IgG, such as IgG1, IgG2, IgG3 or IgG4.

In some embodiments, amino acid changes in the above-mentioned amino acid homology include amino acid substitutions, insertions or deletions. In some embodiments, the amino acid changes in the present disclosure are amino acid substitutions, e.g., conservative substitutions. A conservative substitution refers to the substitution of one amino acid by another amino acid within the same class, for example, one acidic amino acid by another acidic amino acid, one basic amino acid by another basic amino acid, or one neutral amino acid by another neutral amino acid. Exemplary substitutions are shown in the table below (amino acid substitutions).

| Primary residue | Exemplary substitutions | Preferably substitution |
|---|---|---|
| Ala (A) | Val; Leu; Ile | Val |
| Arg (R) | Lys; Gln; Asn | Lys |
| Asn (N) | Gln; His; Asp; Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Nle | Leu |
| Leu (L) | Nle; Ile; Val; Met; Ala; Phe | Ile |
| Lys (K) | Arg; Gln; Asn | Arg |
| Met (M) | Leu; Phe; lle | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Ala; Phe; Nle | Leu |

In preferred embodiments, the amino acid changes in the present disclosure occur in regions outside the CDRs (e.g., in FRs). More preferably, the amino acid changes in the present disclosure occur in the Fc region. In some embodiments, an anti-coronavirus S protein antibody is provided, which comprises an Fc domain comprising one or more mutations that enhance or attenuate binding of the antibody to the FcRn receptor, e.g., at acidic pH as compared to neutral pH.

In some embodiments, the present disclosure encodes antibody CDRs according to the Kabat numbering rules, and the CDRs sequences of specific numbered antibodies are shown in the table below (the HCDRs and LCDRs sequences of neutralizing antibody or an antigen-binding fragment capable of binding to the SARS-CoV-2 virus in this present disclosure)

The CDR sequences of neutralizing antibody or
antigen-binding fragment capable of binding to
the SARS-COV-2 virus

| Antibody | | | | | | |
|---|---|---|---|---|---|---|
| Number | HCDR1 | HCDR2 | HCDR3 | LCDR1 | LCDR2 | LCDR3 |
| ACRO3- | SEQ ID | SEQ ID | SEQ ID | SEQ ID | SEQ ID | SEQ ID |
| 286L | NO. 103 | NO. 104 | NO. 105 | NO. 106 | NO. 107 | NO. 108 |
| | SYYIH | IINPGDGG | AEGSSWL | SGSSSNIG | RNNQRPS | AAWDDGLS |
| | | ASYAQKFQ | GWFDP | SNYVY | | GSGWV |
| | | G | | | | |
| ACRO3- | SEQ ID | SEQ ID | SEQ ID | SEQ ID | SEQ ID | SEQ ID |
| 347K | NO. 127 | NO. 128 | NO. 129 | NO. 130 | NO. 131 | NO. 132 |
| | DVSSLQ | WTVVGTGN | PFCSETS | RASQSVRI | GSSSRAT | QQYDNSPW |
| | | TNYAPRFQ | CSDGFDL | SYLA | | T |
| | | E | | | | |
| ACRO2- | SEQ ID | SEQ ID | SEQ ID | SEQ ID | SEQ ID | SEQ ID |
| 001K | NO. 61 | NO. 62 | NO. 63 | NO. 64 | NO. 65 | NO. 66 |
| | ANYMS | VIYSGGST | VVSDAFD | RASQSISS | KASILES | QHYNSDSY |
| | | NDGDTYYA | I | WLA | | T |
| | | ASVKG | | | | |
| ACRO2- | SEQ ID | SEQ ID | SEQ ID | SEQ ID | SEQ ID | SEQ ID |
| 021L | NO. 67 | NO. 68 | NO. 69 | NO. 70 | NO. 71 | NO. 72 |
| | SFAMT | VISGGAGS | GERPDYG | GGNNIGSK | YDSDRPS | QVWDGSSD |
| | | TYYADSVK | DYFDY | SVH | | HHYV |
| | | G | | | | |
| ACRO2- | SEQ ID | SEQ ID | SEQ ID | SEQ ID | SEQ ID | SEQ ID |
| 028K | NO. 73 | NO. 74 | NO. 75 | NO. 76 | NO. 77 | NO. 78 |
| | SNYMS | VIYSGGST | VVTDAFD | RASQSISS | KASSLES | QQYNSDSY |
| | | YYADSVKG | I | WLA | | T |
| ACRO3- | SEQ ID | SEQ ID | SEQ ID | SEQ ID | SEQ ID | SEQ ID |
| 168L | NO. 79 | NO. 80 | NO. 81 | NO. 82 | NO. 83 | NO. 84 |
| | SSRYY | SLYYSGGT | DHYGDYA | TGSSSNIG | GHNNRPS | QSYDTSLS |
| | WG | YFNPSLKS | LVY | AGYDVH | | ALYV |
| ACRO3- | SEQ ID | SEQ ID | SEQ ID | SEQ ID | SEQ ID | SEQ ID |
| 169L | NO. 85 | NO. 86 | NO. 87 | NO. 88 | NO. 89 | NO. 90 |
| | SSRYY | SLYYSGGT | DHYGDYA | TGSSSNVG | GNNNRPS | QSYDTSLS |
| | WG | YYNPSLKS | LVY | AGYDVH | | ALYV |
| ACRO3- | SEQ ID | SEQ ID | SEQ ID | SEQ ID | SEQ ID | SEQ ID |
| 207L | NO. 91 | NO. 92 | NO. 93 | NO. 94 | NO. 95 | NO. 96 |
| | SYAMN | WINTNTGN | ETSWDG | SGDTLSKQ | KDSERPS | QSADNSGT |
| | | PTYAQGFT | YIY | YTF | | SWV |
| | | G | | | | |
| ACRO3- | SEQ ID | SEQ ID | SEQ ID | SEQ ID | SEQ ID | SEQ ID |
| 283L | NO. 97 | NO. 98 | NO. 99 | NO. 100 | NO. 101 | NO. 102 |
| | ELSMH | GFDPEDAE | GPAIPMA | TGTSSDVG | EVSNRPS | SSYTSSST |
| | | TIYAQKFQ | ATGWFDP | GYNYVS | | YV |
| | | G | | | | |
| ACRO3- | SEQ ID | SEQ ID | SEQ ID | SEQ ID | SEQ ID | SEQ ID |
| 307K | NO. 109 | NO. 110 | NO. 111 | NO. 112 | NO. 113 | NO. 114 |
| | NYAMH | VIWDDGSN | DQGELVT | RASQTISS | TTSSLQS | QQSYSTPS |
| | | KHYADSVK | YFDY | YLN | | WT |
| | | G | | | | |
| ACRO3- | SEQ ID | SEQ ID | SEQ ID | SEQ ID | SEQ ID | SEQ ID |
| 309K | NO. 115 | NO. 116 | NO. 117 | NO. 118 | NO. 119 | NO. 120 |
| | NYDIH | CVGTYGDT | GSVDPTT | RASQGIRN | AASRLES | QQYYSSPP |
| | | YYSDSVKG | NWFFDL | FLA | | IT |
| ACRO3- | SEQ ID | SEQ ID | SEQ ID | SEQ ID | SEQ ID | SEQ ID |
| 341L | NO. 121 | NO. 122 | NO. 123 | NO. 124 | NO. 125 | NO. 126 |
| | SYSMN | YISSSNIT | VLRYYDY | TGTSSDVG | DVSNRPS | SSFTSSST |
| | | IYYADSVK | VWGSYQR | GYDYVS | | LPFV |
| | | G | ELYGMDV | | | |
| ACRO3- | SEQ ID | SEQ ID | SEQ ID | SEQ ID | SEQ ID | SEQ ID |
| 410K | NO. 133 | NO. 134 | NO. 135 | NO. 136 | NO. 137 | NO. 138 |
| | NYGIS | WISSYNGN | ENFNYGD | RASESIGS | HASQSFS | HQSSSLPY |
| | | THYARKLQ | YIIYFDP | SLH | | T |
| | | G | | | | |
| ACRO3- | SEQ ID | SEQ ID | SEQ ID | SEQ ID | SEQ ID | SEQ ID |
| 411L | NO. 139 | NO. 140 | NO. 141 | NO. 142 | NO. 143 | NO. 144 |
| | SGNYY | NIYYSGST | HVFQLLF | SGSSSNIG | SNNQRPS | AAWDDSRN |
| | WG | YYNPSLKS | WDNWFDP | SNSVN | | GVV |
| ACRO3- | SEQ ID | SEQ ID | SEQ ID | SEQ ID | SEQ ID | SEQ ID |
| 414K | NO. 145 | NO. 146 | NO. 147 | NO. 148 | NO. 149 | NO. 150 |
| | RFALH | LISFDGTN | VVSGWHL | RSSQSLLH | LGSNRGS | MQALQTPY |
| | | KFYADSVK | VTYGLDV | TNGYDYLD | | T |
| | | G | | | | |

In some embodiments, the antibody or antigen-binding fragment may further comprise a coupling moiety linked to the polypeptide, the coupling moiety is selected from one or more of radionuclides, drugs, toxins, cytokines, enzymes, fluoresceins, carrier proteins, lipids, and biotin, wherein the polypeptide or antibody and the coupling moiety may be selectively linked by a linker. In some embodiment, the linker is a peptide or a polypeptide.

In some embodiments, the antibody or antigen-binding fragment may be selected from monoclonal antibodies, polyclonal antibodies, antisera, chimeric antibodies, humanized antibodies, and human antibodies; More preferably, the antibody is selected from the group consisting of multispecific antibodies, single chain Fv (scFv), single chain antibodies, anti-idiotypic (anti-Id) antibodies, diabodies, minibodies, nanobodies, single domain antibodies, Fab fragments, F(ab′) fragments, disulfide-linked bispecific Fv (sdFv) and intrabodies.

In some embodiments, the antibodies or antigen-binding fragments in the present disclosure can be produced by recombinant expression. The nucleic acids encoding light and heavy chain variable regions, optionally linked to constant regions, can be inserted into expression vectors. Vectors comprising nucleic acids encoding antibodies are provided in an aspect of the present disclosure. Nucleic acids encoding light and heavy chains can be cloned into the same or different expression vectors. Nucleic acids encoding the antibody chains in the present disclosure can be operably linked to one or more regulatory sequences in the expression vector to ensure expression of the antibody chains. Expression regulatory sequences include, but are not limited to, promoters (e.g., naturally associated or heterologous promoters), signal sequences, enhancer elements, and transcription termination sequences. Preferably, the expression regulatory sequence is a eukaryotic promoter system in a vector capable of transforming or transfecting eukaryotic host cells. Such vectors can be incorporated into a suitable host whereby the host is maintained under conditions suitable for high level expression of the nucleotide sequence and collection and purification of the antibody.

2. Nucleic acids of the present disclosure, vectors that comprise the nucleic acid, and host cells.

The present disclosure provides nucleic acid encoding any of the above coronavirus S protein antibodies or antigen-binding fragments or any chain thereof.

In an embodiment, a vector comprising the nucleic acid is provided. In an embodiment, the vector is an expression vector. In an embodiment, a host cell comprising the nucleic acid or the vector is provided. In an embodiment, the host cell is eukaryotic. In another embodiment, the host cell is selected from yeast cells, mammalian cells, or other cells suitable for the production of antibodies or antigen-binding fragments. In another embodiment, the host cell is prokaryotic.

The nucleic acid involved in the present disclosure is a nucleic acid encoding an anti-SARS-CoV-2 S protein antibody or its antigen-binding fragment or its VH or VL domain. It can be understood that any nucleic acid capable of encoding the above antibody or its antigen-binding fragment or its VH or VL domain is within the scope of the present disclosure.

In some specific embodiments, the nucleic acid sequences are shown in the following table (the specific nucleic acid sequences encoding the neutralizing antibody or antigen-binding fragment that can bind to the SARS-CoV-2 virus in the present disclosure)

| Anti- | | SEQ | |
|---|---|---|---|
| bodies | | NO. | Corresponding DNA Sequence |
| ACRO3- | VH | 29 | CAGGTGCAGCTGGTGCAGTCTGGGGCTGAGGTGAAGAAGCCT |
| 286L | | | GGGGCCTCAGTGAAGGTTTCCTGCAAGGCATCTGGATACACC |
| | | | TTCTCCAGCTACTATATACACTGGGTGCGACAGGCCCCTGGA |
| | | | CAGCAAGTTACGCACAGAAATTTCAGGGCAGAGTCACCCTGA |
| | | | CCAGGGACACGTCCACGAGCACACTTTACATGGAGCTGAGCA |
| | | | GCCTGAGATCTGAGGATACGGCCGTGTATTACTGTGCGAGAG |
| | | | CCGAGGGCAGCAGCTGGCTTGGGTGGTTCGACCCCTGGGGCC |
| | | | AGGGAACCCTGGTCACCGTCTCCTCA |
| | VL | 31 | CAGTCTGTGCTGACTCAGCCACCCTCAGCGTCTGGGACCCCC |
| | | | GGGCAGAGGGTCACCATCTCTTGTTCTGGAAGCAGCTCCAAC |
| | | | ATCGGAAGTAATTATGTATACTGGTACCAGCAGCTCCCAGGA |
| | | | ACGGCCCCCAAACTCCTCATCTATAGGAATAATCAGCGGCCC |
| | | | TCAGGGGTCCCTGACCGATTCTCTGGCTCCAGGTCTGGCACC |
| | | | TCAGCCTCCCTGGCCATCAGTGGGCTCCGGTCCGAGGATGAG |
| | | | GCTGATTATTACTGTGCAGCATGGGATGACGGCCTGAGTGGT |
| | | | TCGGGGTGGGTGTTCGGCGGAGGGACCAAGCTGACCGTGCTG |
| ACRO3- | VH | 45 | CAAATGCAGCTGGTGCAGTCTGGGCCTGAGGTGAAGAAGCCT |
| 347K | | | GGGACCTCAGTGAAGGTCTCCTGCAAGGCTTCTGGATTCACC |
| | | | TTTACTGACGTCTCTTCTCTGCAATGGGTGCGACAGGCTCGT |
| | | | GGACAACGCCTTGAGTGGATAGGATGGACGGTCGTTGGCACT |
| | | | GGTAATACAAACTACGCACCGAGGTTCCAAGAAAGAGTCACC |
| | | | ATTACCACGGACAAGTCCACAAGCACGGCCTACATGGAACTG |
| | | | AGCAGCCTGAGATCCGAGGACACGGCCGTGTATTACTGTGCG |
| | | | GCACCGTTTTGTAGTGAGACTTCCTGTAGTGATGGCTTTGAT |
| | | | CTGTGGGGCCAAGGGACAAAGGTCACCGTCTCTTCA |
| | VL | 47 | GAAATTGTGTTGACGCAGTCTCCAGGCACCCTGTCTTTGTCT |
| | | | CCAGGGGACAGAGCCACCCTCTCATGCAGGGCCAGTCAGAGT |
| | | | GTTAGGATCTCCTACTTAGCCTGGTACCAGCAGAAACCTGGC |
| | | | CAGGCTCCCAGGCTCCTCATCTCTGGTTCGTCCAGTAGGGCC |
| | | | ACTGGCATCCCAGACAGGTTCAGTGCCAGTGGGTCTGGGACA |
| | | | GACTTCGCTCTCACCATCAGCAGACTGGAGCCTGAAGATTTT |
| | | | GCAGTATATTACTGTCAGCAGTATGATAACTCACCGTGGACG |
| | | | TTCGGCCAAGGGACCAAAGTGGAAGTCAAA |
| ACRO2- | VH | 1 | GAGGTGCAGCTGGTGGAGTCTGGAGGAGGCGTGATCCAGCCT |
| 001K | | | GGGGGGTCCCTGAGACTCTCCTGTGCAGCCTCTGGGTTCACC |
| | | | GTCAGTGCCAACTACATGAGCTGGGTCCGCCAGGCTCCAGGG |
| | | | AAGGGGCTGGAGTGGGTCTCAGTTATTTATAGCGGTGGCAGC |
| | | | ACAAACGATGGTGACACATACTACGCAGCCTCCGTGAAGGGC |
| | | | CGATTCACCATCTCCAGAGACAATTCCAAGAACACGCTGTAT |
| | | | CTTCAAATGAACAGCCTGAGAGCCGAGGACACGGCCGTGTAT |
| | | | TACTGTGCGAGAGTGGTGTCTGATGCTTTTGATATCTGGGGC |
| | | | CAAGGGGCAATGGTCACCGTGTCTTCT |
| | VL | 3 | GACATCCAGATGACCCAGTCTCCTTCCACCCTGTCTGCATCT |
| | | | GTAGGAGACAGAGTCACCATCACTTGCCGGGCCAGTCAGAGT |
| | | | ATTAGTAGCTGGTTGGCCTGGTATCAGCAGAAACCAGGGAAA |
| | | | GCCCCTAAGCTCCTGATCTATAAGGCATCTATTTTAGAAAGT |
| | | | GGGGTCCCATCAAGGTTCAGCGGCAGTGGATCTGGGACAGAA |
| | | | TTCACTCTCACCATCAGCAGCCTGCAGCCTGATGATTTTGCA |
| | | | ACTTATTACTGCCAACACTATAATAGCGATTCCTACACTTTT |
| | | | GGCCAGGGGACCAAGCTGGAGATCAAA |
| ACRO2- | VH | 5 | GAGGTGCAGCTGTTGGAGTCTGGGGGAGGCTTGGTACAGCCT |
| 021L | | | GGGGGGTCCCTGAGACTCTCCTGTGCAGCCTCTGGATTCACC |
| | | | TTTAGCAGCTTTGCCATGACCTGGGTCCGCCAGGCTCCAGGG |
| | | | AAGGGGCTGGAGTGCGTCTCAGTTATTAGTGGTGGTGCTGGT |
| | | | AGCACATACTACGCAGACTCCGTGAAGGGCCGGTTCACCCTC |
| | | | TCCAGAGACAATTCCAAGAACACGCTGTATCTGCAAATGAAC |
| | | | AGCCTGAGAGCCGAGGACACGGCCGTATATTACTGTGCGAAA |
| | | | GGGGAGAGGCCCGACTACGGTGACTACTTTGACTACTGGGGC |
| | | | CAGGGAACCCTGGTCACCGTCTCCTCA |
| | VL | 7 | TCTTATGTGCTGACTCAGCCCCCCTCAGTGTCAGTGGCCCCA |
| | | | GGAAAGACGGCCAGGATTACCTGTGGGGGAAACAACATTGGA |
| | | | AGTAAAAGTGTGCACTGGTACCAGCAGAGGCCAGGCCAGGCC |
| | | | CCTGTCCTGGTCATGTATTATGATAGCGACCGGCCCTCAGGG |
| | | | ATCCCTGACCGATTCTCTGGCTCCAACTCTGGGAACACGGCC |
| | | | ACCCTGACCATCAGCCGGGTCGAAGCCGGGGATGAGGCCGAC |
| | | | TATTACTGTCAGGTGTGGGATGGTAGTAGTGATCATCATTAT |
| | | | GTCTTCGGCACTGGGACCAAGCTGACAGTGCTG |
| ACRO2- | VH | 9 | GAGGTGCAGCTGGTGGAGTCTGGAGGAGGCTTGGTCCAGCCT |
| 028K | | | GGGGGGTCCCTGAGACTCTCCTGTGCAGCCTCTGGGTTCAGC |
| | | | GTCAGTAGCAACTACATGAGCTGGGTCCGCCAGGCTCCAGGG |
| | | | AAGGGGCTGGAGTGGGTCTCAGTTATTTATAGCGGTGGTAGC |
| | | | ACATACTACGCAGACTCCGTGAAGGGCCGATTCACCATCTCC |
| | | | AGACACAATTCCAAGAACACGCTGTATCTTCAAATGAACAGC |
| | | | CTGAGAGCTGAGGACACGGCCGTGTATTACTGTGCGAGAGTC |
| | | | GTAACCGACGCTTTTGATATCTGGGGCCAAGGGACAATGGTC |
| | | | ACCGTCTCTTCA |
| | VL | 11 | GACATCCAGATGACCCAGTCTCCTTCCACCCTGTCTGCATCT |
| | | | GTTGGAGACAGAGTCACCATCACTTGCCGGGCCAGTCAGAGT |
| | | | ATTAGTAGCTGGTTGGCCTGGTATCAGCAGACACCAGGGAAA |
| | | | GCCCCTAAGCTCCTGATCTATAAGGCATCTAGTTTAGAAAGT |
| | | | GGGGTCCCATCAAGGTTCAGCGGCAGTGGATCTGGGACAGAA |
| | | | TTCACTCTCACCATCAGCAGCCTGCAGCCTGATGATTTTGCA |
| | | | ATTTATTACTGCCAACAGTATAATAGTGATTCGTACACTTTT |
| | | | GGCCAGGGGACCAAGCTGGAGATCAAA |
| ACRO3- | VH | 13 | CAGCTGCAGCTGCAGGAGTCGGGCCCAGGACTGGTGAAGCCT |
| 168L | | | TCGGAGACCCTGTCCCTCACCTGCACTGTCTCTGGTGGCTCC |
| | | | ATCAGCAGTAGTCGTTACTACTGGGGCTGGATCCGCCAGCCC |
| | | | CCAGGGAAGGGGCTGGAGTGGATTGGGAGTCTCTATTATAGT |
| | | | GGGGGCACCTACTTCAACCCGTCCCTCAAGAGTCGAGTCACC |
| | | | ATCTCAGTAGACACGTCCAAGAACCAGTTCTCCCTGAAACTG |
| | | | ACCTCTGTGACCGCCGCGGACACGGCCGTTTATTACTGTGCG |
| | | | AGAGACCATTACGGTGACTACGCCCTTGTCTACTGGGGCCAG |
| | | | GGAACCCTGGTCACCGTCTCCTCA |
| | VL | 15 | CAGTCTGTGCTGACGCAGCCGCCCTCAGTGTCTGGGGCCCCA |
| | | | GGGCAGAGGGTCACCATCTCCTGCACTGGGAGCAGCTCCAAC |
| | | | ATCGGGGCAGGTTATGATGTACACTGGTACCAGCAGCTTCCA |
| | | | GGAACAGCCCCCAAACTCCTCATCTATGGTCACAACAATCGG |
| | | | CCCTCAGGGGTCCCTGACCGATTCTCTGGCTCCCAGTCTGGC |
| | | | ACCTCAGCCTCCCTGGCCATCACTGGGCTCCAGGCTGAGGAT |
| | | | GAGGCTGATTATTACTGCCAGTCCTATGACACTAGCCTGAGT |
| | | | GCCCTTTATGTCTTCGGAACTGGGACCAAGGTCACCGTCCTG |
| ACRO3- | VH | 17 | CAGCTGCAGCTGCAGGAGTCGGGCCCAGGACTGGTGAAGCCT |
| 169L | | | TCGGAGACCCTGTCCCTCACCTGCACTGTCTCTGGTGGCTCC |
| | | | ATCAGCAGTAGTCGTTACTACTGGGGCTGGATCCGCCAGCCC |
| | | | CCAGGGAAGGGGCTGGAGTGGATTGGGAGTCTGTATTATAGT |
| | | | GGGGGCACCTACTACAACCCGTCCCTCAAGAGTCGAGTCACC |
| | | | ATCTCAGTAGACACGTCCAAGAACCAGTTCTCCCTGAAACTG |
| | | | ACCTCTGTGACCGCCGCGGACACGGCCGTTTATTACTGTGCG |
| | | | AGAGACCATTACGGTGACTACGCCCTTGTCTACTGGGGCCAG |
| | | | GGAACCCCAGTCACCGTCTCCTCA |
| | VL | 19 | CAGTCTGTGCTGACGCAGCCGCCCTCAGTGTCTGGGGCCCCA |
| | | | GGGCAGAGGGTCACCATCTCCTGCACTGGGAGCAGCTCCAAC |
| | | | GTCGGGGCAGGTTATGATGTACACTGGTACCAGCAGCTTCCA |
| | | | GGAACAGCCCCCAAACTCCTCATCTATGGTAACAACAATCGG |
| | | | CCCTCAGGGGTCCCTGACCGATTCTCTGGCTCCCAGTCTGGC |
| | | | ACCTCAGCCTCCCTGGCCATCACTGGGCTCCAGACTGAGGAT |
| | | | GAGGCTGATTATTACTGCCAGTCCTATGACACTAGCCTGAGT |
| | | | GCCCTTTATGTCTTCGGAACTGGGACCAAGGTCACCGTGCTG |
| ACRO3- | VH | 21 | CAGGTGCAGCTGGTGCAATCTGGGTCTGAGTTGAAGAAGCCT |
| 207L | | | GGGGCCTCAGTGAAGGTTTCCTGCAAGGCCTCTGGATACAGC |
| | | | TTCACTAGTTATGCTATGAATTGGGTGCGACAGGCCCCTGGA |
| | | | CAAGGGCTTGAGTGGATGGGATGGATCAATACCAATACTGGG |
| | | | AACCCAACGTATGCCCAGGGCTTCACAGGACGGTTTGTCTTC |
| | | | TCCTTGGACACCTCTGTCAACACGGCATATCTGCAGATCAGC |
| | | | GGCCTGAAGGCTGAGGACACTGCCGTCTATTACTGTGCGAGA |
| | | | GAGACCAGTTGGGATGGCTATATCTATTGGGGCCAGGGAACC |
| | | | CTGGTCACCGTCTCCTCA |
| | VL | 23 | TCCTATGAGGTGACACAGCCACCCTCGGTGTCAGTGTCCCCA |
| | | | GGACAGACGGCCAGGATCACCTGCTCTGGAGATACATTGTCA |
| | | | AAGCAATATACTTTTTGGTACCAACAGAAGACAGGCCAGGCC |
| | | | CCTGTGTTGGTGATATATAAAGACAGTGAGAGGCCCTCAGGG |
| | | | ATCCCTGAGCGATTCTCTGGCTCCAGCTCAGGGACAACAGTC |
| | | | ACCTTGACCATCAGTGGAGTCCAGGCAGAAGACGAGGCTGAC |
| | | | TATTATTGTCAATCAGCAGACAACAGTGGTACTTCTTGGGTG |
| | | | TTCGGCGGAGGGACCAAGCTGACAGTGCTG |
| ACRO3- | VH | 25 | CAGGTCCAGCTGGTACAGTCTGGGGCTGAGGTGAAGAAGCCT |
| 283L | | | GGGGCCTCAGTGAAGGTCTCCTGCAAGGTTTCCGGATACACC |
| | | | CTCATTGAATTATCCATGCACTGGGTGCGACAGGCTCCTGGA |
| | | | AAAGGGCTTGAGTGGATGGGAGGTTTTGATCCTGAAGATGCT |
| | | | GAAACAATCTACGCACAGAAGTTCCAGGGCAGAGTCACCATG |
| | | | ACCGAGGACACATCTACAGACACAGCCTATATGGAGCTGAGC |
| | | | AGGCTGAGATCTGAGGACACGGCCGTGTATTACTGTGCAATA |
| | | | GGTCCCGCGATACCAATGGCTGCTACTGGGTGGTTCGACCCC |
| | | | TGGGGCCAGGGAACCCTGGTCACCGTCTCCTCA |
| | VL | 27 | CAGTCTGCCCTGACTCAGCCTGCCTCCGTGTCTGGGTCTCCT |
| | | | GGACAGTCGATCACCATCTCCTGCACTGGAACCAGCAGTGAC |
| | | | GTTGGTGGTTATAACTATGTCTCCTGGTACCAACAGCACCCA |
| | | | GGCAAAGCCCCCAAACTCATGATTTATGAGGTCAGTAATCGG |
| | | | CCCTCAGGGGTTTCTAATCGCTTCTCTGGCTCCAAGTCTGGC |
| | | | AACACGGCCTCCCTGACCATCTCTGGGCTCCAGGCTGAGGAC |
| | | | GAGGCTGATTATTACTGCAGCTCATATACAAGCAGCAGCACT |
| | | | TATGTCTTCGGAACTGGGACCAAGGTCACCGTGCTG |
| ACRO3- | VH | 33 | CAGGTGCAGCTGGTGGAGTCTGGGGGAGGCGTGGTCCAGCCT |
| 307K | | | GGGAGGTCCCTGAGACTCTCCTGTTCAGCGTCTGGATTCATC |
| | | | TTCAGTAATTATGCCATGCACTGGGTCCGCCAGGCCCCAGGC |
| | | | AAGGGGCTGGAGTGGGTGGCAGTTATATGGGATGATGGAAGT |
| | | | AATAAACACTATGCAGACTCCGTGAAGGGCCGATTCACCATC |
| | | | TCCAGAGACAATTCCAAGAACACGCTTTATCTGCAAATGAAC |
| | | | AGCCTGAGAGCCGAGGACACGGCTGTGTATTACTGTGCGAGA |
| | | | GATCAAGGGGAGCTAGTTACTTACTTTGACTACTGGGGCCAG |
| | | | GGAACCCTGGTCATCGTCTCTTCA |
| | VL | 35 | GACATCCAGATGACCCAGTCTCCATCCTCCCTGTCTGCATCT |
| | | | GTAGGAGACAGAGTCACCATCACTTGCCGGGCAAGTCAGACC |
| | | | ATTAGCAGCTATTTAAATTGGTATCAGCAGAAACCAGGGAAA |
| | | | GCCCCTAAACTCCTGATCTATACTACATCCAGTTTGCAAAGT |
| | | | GGGGTTCCATCACGGTTCAGTGGCAGTGGATCTGGGACAGAT |
| | | | TTCACTCTCACCATCAGCAGTCTGCAACCTGAAGATTTTGCA |
| | | | ACTTACTACTGTCAACAGAGTTACAGTACCCCTTCGTGGACG |
| | | | TTCGGCCAAGGGACCAAGGTGGAAATCAAA |
| ACRO3- | VH | 37 | GAGGTGCAGCTGGTGGAGTCTGGGGGAGGCTTGGTACAGCCG |
| 309K | | | GGGGGGTCCCTGAGACTCTCCTGTGGAGCCTCTGGATTCAGC |
| | | | TTCAGTAACTACGACATACACTGGGTCCGCCAAGCTATAGGA |
| | | | ACAGGTCTGGAGTGGGTCTCATGTGTTGGTACTTATGGTGAC |
| | | | ACATACTATTCAGACTCCGTGAAGGGCCGATTCACCATCTCC |
| | | | AGAGAAAATGCCAAGAACTCCTTGTATCTTCAAATGAACAGC |
| | | | CTGAGACTCGGAGACACGGCTGTATATTTCTGTGCAAGAGGC |
| | | | TCGGTAGACCCTACGACCAACTGGTTCTTCGATCTCTGGGGC |
| | | | CGTGGCACCCTGGTCACTGTCTCCTCA |
| | VL | 39 | GACATCCAGATGACCCAGTCTCCATCCTCCCTGTCTGCATCT |
| | | | GTAGGAGACAGAGTCACCATCACTTGCCGGGCGAGTCAGGGC |
| | | | ATTAGGAATTTTTTGGCCTGGTATCAGCAGAAACCAGGGAAA |
| | | | GCCCCTAAACTCCTACTCTCTGCTGCATCCAGATTGGAAAGT |
| | | | GGGGTCCCATCGAGGTTCAGTGGCAGTGGATCTGGGACGGAT |
| | | | TACACTCTCACCATCAGCAGCCTGCAGCCTGAAGATTTTGCA |
| | | | ACTTATTACTGTCAACAGTATTATAGTTCCCCTCCGATCACC |
| | | | TTCGCCCAAGGGACACGACTGGAGATTAAA |
| ACRO3- | VH | 41 | GAGGTGCAGCTGGTGGAGTCTGGGGGAGGCTTGGTACAGCCT |
| 341L | | | GGGGGGTCCCTGAGACTCTCCTGTGCAGCCTCTGGATTCACC |
| | | | TTCAGTAGCTATAGCATGAACTGGGTCCGCCAGGCTCCAGGG |
| | | | AAGGGGCTGGAGTGGGTTTCATACATTAGTAGTAGTAATATT |
| | | | ACCATATACTACGCAGACTCTGTGAAGGGCCGATTCACCATC |
| | | | TCCAGAGACAATGCCAAGAACTCACTGTATATGCAAGTGAAC |
| | | | AGCCTGAGAGACGAGGACACGGCTGTGTATTACTGTGCGAGA |
| | | | GTCCTACGGTATTATGATTACGTTTGGGGGAGTTATCAACGG |
| | | | GAACTCTACGGTATGGACGTCTGGGGCCAAGGGACCACGGTC |
| | | | ACCGTCTCCTCA |
| | VL | 43 | CAGTCTGCCCTGACTCAGCCTGCCTCCGTGTCTGGGTCTCCT |
| | | | GGACAGTCGATCACCATCTCCTGCACTGGAACCAGCAGTGAC |
| | | | GTTGGTGGTTATGACTATGTCTCCTGGTACCAGCAACACCCC |
| | | | GGCAAAGCCCCCAAACTGATGATTTATGATGTCAGTAATCGG |
| | | | CCCTCAGGGGTTTCTAATCGCTTCTCTGGCTCCAAGTCTGGC |
| | | | AACACGGCCTCCCTGACCATCTCTGGGCTCCAGGCTGAGGAC |
| | | | GAGGCTGATTATTACTGCAGCTCATTTACATCCAGCAGCACT |
| | | | CTCCCCTTTGTCATCGGAACTGGGACCAAGGTCACCGTCCTA |
| ACRO3- | VH | 49 | CAGGTTCAGCTGGTGCAGTCTGGGGCTGAGGTGAAGAAGCCT |
| 410K | | | GGGGCCTCAGTGAAGGTCTCCTGCAAGGCTTCTGGTTACACC |
| | | | TTTACCAACTACGGTATCAGCTGGGTGCGACAGGCCCCTGGA |
| | | | CAAGGGCTTGAGTGGATGGGATGGATCAGCAGTTACAATGGT |
| | | | AATACACACTATGCACGGAAGCTCCAGGGCAGAGTCACCATG |
| | | | ACCACAGACACATCCACGAGCACAGTCTACATGGAGCTGAGG |
| | | | AGCCTGAGATCTGACGACACGGCCGTGTATTACTGTGCGAGA |
| | | | GAAAATTTTAACTACGGTGACTACATCATTTACTTCGACCCC |
| | | | TGGGGCCAGGGAACCCTGGTCACCGTCTCCTCA |
| | VL | 51 | GAAATTGTGCTGACTCAGTCTCCAGACTTTCAGTCTGTGACT |
| | | | CCAAAGGAGAAAGTCACCATCACCTGCCGGGCCAGTGAAAGC |
| | | | ATTGGTAGTAGCTTACACTGGTACCAGCAGAAACCAGATCAG |
| | | | TCTCCAAAACTCCTCATCAAGCATGCTTCCCAGTCCTTCTCA |
| | | | GGGGTCCCCTCGAGGTTCAGTGGCAGTGGATCTGGGACAGAT |
| | | | TTCACCCTCACCATCAATGGCCTGGAAGCTGAAGATGCTGCA |
| | | | ACGTATTACTGTCATCAGAGTAGTAGTTTACCGTACACTTTT |
| | | | GGCCAGGGGACCAAGCTGGAGATCAAA |
| ACRO3- | VH | 53 | CAGCTGCAGCTGCAGGAGTCGGGCCCAGGACTGGTGAAGCCT |
| 411L | | | TCGGAGACCCTGTCCCTCACCTGCACTGTCTCTGGTGGCTCC |
| | | | ATCAGCAGTGGGAATTACTACTGGGGCTGGATCCGCCAGCCC |
| | | | CCAGGGAAGGGGCTGGAGTGGATCGGAAATATCTATTACAGT |
| | | | GGGAGCACCTACTACAACCCGTCCCTCAAGAGTCGAGTCACC |
| | | | CTCTCCGTCGACACGCCCAAGAACCAGTTCTCCCTGAAGCTG |
| | | | AGCTCTGTGACCGCCGCAGACACGGCTGTGTATTACTGTGCG |
| | | | AGACATGTGTTCCAGCTGCTATTTTGGGACAACTGGTTCGAC |
| | | | CCCTGGGGCCAGGGAACCCTGGTCACCGTCTCCTCA |
| | VL | 55 | CAGTCTGTGCTGACTCAGCCACCCTCAGCGTCTGGGACCCCC |
| | | | GGGCAGAGGGTCACCATCTCTTGTTCTGGAAGCAGCTCCAAC |
| | | | ATCGGAAGTAATAGTGTAAACTGGTACCAGCAGATCCCAGGA |
| | | | CCGGCCCCCAAACTCCTCATCCATAGTAATAATCAGCGGCCC |
| | | | TCAGGGGTCCCTGACCGATTCTCTGGCTCCAAGTCAGGCACC |
| | | | TCAGCCTCCCTGACCATCAGCGGGATCCAGTCTGAGGATGAG |
| | | | GCTGATTATTACTGTGCAGCATGGGACGACAGCAGGAATGGT |
| | | | GTGGTATTCGGCGGAGGGACCAAGCTGACCGTGCTG |
| ACRO3- | VH | 57 | CAGGTGCAGCTGGTGGAGTCTGGGGGAGGCGTGGTCCAGCCT |
| 414K | | | GGGAAGTCCCTGAGAGTCTCCTGTGCAGCCTCTGGATTCCCC |
| | | | TTCAGTAGATTTGCCTTACACTGGGTCCGCCAGGCTCCAGGC |
| | | | AAGGGGCTGGAGTGGGTGGCACTTATATCGTTTGATGGAACT |
| | | | AATAAGTTCTATGCAGACTCCGTGAAGGGCCGATTCACCATC |
| | | | TCCAGAGACAATTCCAAGAACATTATGTATTTGGACATGAAC |
| | | | AGCCTGGGAGACGAAGACACGGCTGTGTATTATTGTGCGAAA |
| | | | GTTGTTAGTGGCTGGCACTTAGTGACCTACGGTTTGGACGTC |
| | | | TGGGGCCAAGGGACCACGGTCACCGTCTCCTCA |
| | VL | 59 | GATATTGTGATGACTCAGTCTCCGCTCTCCCTGTCCGTCACC |
| | | | CCTGGAGAGCCGGCCTCCATCTCTTGTAGGTCAAGTCAGAGC |
| | | | CTCCTGCATACTAATGGGTACGATTATTTGGATTGGTACCTG |
| | | | CAGAAGCCAGGGCAGTCTCCACAGCTCCTGATGTACTTGGGT |
| | | | TCTAATCGGGGCTCCGGGGTCCCTGTCAGGTTCAGTGCCAGT |
| | | | GGAACAGGCACAGATTTCACACTGAAAATCAGCAGAGTGGAG |
| | | | GCTGAGGATGTTGGGGTTTATTACTGCATGCAAGCTCTACAA |
| | | | ACTCCGTACACTTTTGGCCAGGGGACCAAGCTGGAGATCAAA |

The nucleic acid of the present disclosure may also be a nucleic acid sequence having codon degeneracy with any of the above-mentioned sequences, such as a sequence having at least 90%, 91%, 92%, 93%, 94%, 95%, 96%, 97%, 98% or 99% identity with the above-mentioned sequences.

In the present disclosure, the vector comprising one or more nucleic acids encoding the antibodies may be a cloning vector or an expression vector, without limitation.

In an embodiment, the vector is an expression vector, such as a eukaryotic expression vector. The vectors include, but are not limited to, viruses, plasmids, cosmids, bacteriophage lambda, or yeast artificial chromosomes (YACs), etc.

In an embodiment, the vector comprises optional regulatory sequences. In some embodiments, the regulatory sequence may be selected from a leader sequence, a polyadenylation sequence, a propeptide sequence, a promoter, a signal sequence, a transcription terminator, or any combination thereof, without limitation.

The host cells comprising the expression vectors of the present disclosure, may include, for example, yeast cells, mammalian cells, or other cells suitable for preparing antibodies or antigen-binding fragments. In some embodiments, suitable host cells include prokaryotic microorganisms, such as E. coli. The host cells can also be eukaryotic microorganisms such as filamentous fungi or yeast, or various eukaryotic cells such as insect cells and the like. Vertebrate cells can also be used as hosts. For example, mammalian cell lines adapted for growth in suspension can be used. Examples of useful mammalian host cell lines comprise the SV40 transformed monkey kidney CV1 line (COS-7); Human embryonic kidney (HEK293 or 293F cells), 293 cells, baby hamster kidney cells (BHK), monkey kidney cells (CV1), African green monkey kidney cells (VERO-76), human cervical cancer cells (HELA), canine kidney cells (MDCK), Buffalo rat liver cells (BRL 3A), human lung cells (W138), human liver cells (Hep G2), Chinese hamster ovary cells (CHO cells), CHOS cells, NSO cells, myeloma cell lines such as YO, NSO, P3X63 and Sp2/0, etc. For a review of mammalian host cell lines suitable for protein production see, e.g., Yazaki and Wu, Methods in Molecular Biology, Vol. 248 (ed. B. K. C. Lo, Humana Press, Totowa, NJ), pp. 255-268 (2003). In a preferred embodiment, the host cells are CHO cells or 293 cells.

The vectors in the present disclosure comprising polynucleotide sequences of interest (e.g., heavy and light chain-coding sequences and expression regulatory sequences) can be transferred into host cells by well-known methods, which vary depending on the type of cellular host. For example, calcium chloride transfection is commonly used in prokaryotic cells, while calcium phosphate treatment, electroporation, lipofection, and biolistic or virus-based transfection can be used in other cellular hosts. (See generally Green and Sambrook, Molecular Cloning: A Laboratory Manual (Cold Spring Harbor Press, 4th ed., 2012). Other methods for transforming mammalian cells comprise the use of polybrene, protoplast fusion, liposomes, electroporation, and microinjection (see generally, Sambrook et al., supra). To generate transgenic animals, the transgene can be microinjected into fertilized oocytes, or can be integrated into the genome of embryonic stem cells, and the nuclei of these cells are transferred into enucleated oocytes.

3. Preparation, production, and purification of the antibody or antigen-binding fragment of the present disclosure.

The method for preparing the antibody or the antigen-binding fragment of the present disclosure may include expressing the vector in host cell culture to produce the antibody and recovering the antibody from the cell culture.

In some embodiments, the method may comprise transferring a vector comprising one or more nucleic acids encoding the anti-SARS-CoV-2 S protein antibody or antigen-binding fragment as described above into a host cell as in the present disclosure, cultivating the host cell culture under conditions that allow expression of the nucleic acids, and recovering the expressed anti-SARS-CoV-2 S protein antibody or antigen-binding fragment. Any suitable method known in the art may be used.

The present disclosure provides a method for preparing an anti-coronavirus antibody or an antigen-binding fragment, wherein the method comprises culturing a host cell comprising a nucleic acid encoding the antibody or an expression vector comprising the nucleic acid under conditions suitable for expressing the nucleic acid encoding the anti-coronavirus antibody or antigen-binding fragment, and optionally isolating the antibody or antigen-binding fragment. In some embodiments, the method further comprises recovering and purifying the corresponding antibody or antigen-binding fragment from the host cell (or host cell culture medium).

In some embodiments, the antibodies or antigen-binding fragments can be purified by known art techniques such as high performance liquid chromatography, ion exchange chromatography, gel electrophoresis, affinity chromatography, size exclusion chromatography, and the like. The actual conditions used to purify a particular protein may also depend on such factors as net charge, hydrophobicity, and hydrophilicity, and may be apparent to those skilled in the art. The purity of the antibodies of the present disclosure can be determined by any of a variety of well-known analytical methods, comprising size exclusion chromatography, gel electrophoresis, high performance liquid chromatography, and the like.

In some embodiments, the anti-coronavirus antibodies provided in the present disclosure can be identified, screened or characterized for their physical/chemical properties and/or biological activities by various assays known in the art. On one hand, the antibodies of the present disclosure are tested for their antigen-binding activity, for example, by known methods such as ELISA, Western blotting and the like. Methods known in the art can be used to determine the binding ability of the antibody to the coronavirus S protein. In some embodiments, SPR or biofilm layer interference can be used to determine the binding of the anti-coronavirus S protein antibody of the present disclosure to the coronavirus S protein.

4. The pharmaceutical composition and therapeutic use of the present disclosure.

In some embodiments, the present disclosure provides a composition comprising any of the anti-coronavirus S protein antibodies or antigen-binding fragments. In some embodiments, the composition is a pharmaceutical composition, and the pharmaceutical composition may be for preventive or therapeutic purposes. It is understood that the composition may include vaccine-like compositions.

In an embodiment, the composition further comprises pharmaceutical excipients. In an embodiment, the composition (e.g., pharmaceutical composition) comprises a combination of the anti-coronavirus S protein antibody or antigen-binding fragment of the present disclosure and one or more other therapeutic agents (e.g., anti-infection active agents, small molecule drugs). The anti-infective active agent and small molecule drugs are any anti-infective active agent and small molecule drug used to treat, prevent or alleviate coronavirus infection in subjects, including but not limited to remdesivir, ribavirin, oseltamivir, zanamivir, hydroxychloroquine, interferon-α2b, analgesics, azithromycin, and corticosteroids.

In the present disclosure, coronavirus infection comprises infection caused by coronavirus (including but not limited to 2019-n CoV, SARS-CoV).

In some embodiments, the anti-SARS-CoV-2 S protein antibody or antigen-binding fragment can be formulated and/or administered as a pharmaceutical composition comprising an active therapeutic antibody agent and various other pharmaceutically acceptable ingredients. The preferred form depends on the intended mode of administration and therapeutic use. Depending on the desired formulation, the composition may also contain pharmaceutically acceptable, non-toxic carriers or diluents, which are defined as carriers commonly used in formulating pharmaceutical compositions for animal or human administration. The diluent is chosen so as not to affect the biological activity of the combination. Examples of such diluents are distilled water, physiological phosphate-buffered saline, Ringer's solution, dextrose solution, and Hank's solution. In addition, the pharmaceutical composition or preparation may also include other carriers, adjuvants or non-toxic, non-therapeutic, non-immunogenic stabilizers and the like.

In some embodiments, the pharmaceutical composition of the anti-SARS-CoV-2 S protein antibody or antigen-binding fragment may also comprise large, slowly metabolized macromolecules, such as proteins, polysaccharides, such as chitosan, polylactic acid, polyglycolic acid, and copolymers (such as agarose, cellulose, etc.), polymeric amino acids, amino acid copolymers, and lipid aggregates (such as oil droplets or liposomes). In addition, these carriers can act as immunostimulants (i.e., adjuvants).

In an embodiment, the pharmaceutical compositions of the present disclosure may also comprise more than one active ingredient which are necessary for treating a specific condition, preferably those with complementary activities that do not have any negative effects on each other. For example, it is desirable to also provide other anti-infective active ingredients, such as other antibodies, anti-infective active agents, small molecule drugs or immunomodulatory agent, and the like. The active ingredients are suitably present in combination in amounts effective for the intended use.

In some embodiments, the anti-infective active agent and the small molecule drug are any anti-infective active agent and small molecule drug used to treat, prevent or alleviate coronavirus infection in a subject, including but not limited to remdesivir, ribavirin, oseltamivir, zanamivir, hydroxychloroquine, interferon-α2b, analgesics, azithromycin, and corticosteroids.

The term “pharmaceutical carrier” may include any and all solvents, dispersion media, isotonic and absorption delaying agents, and the like that are physiologically compatible. Pharmaceutical carriers suitable for use in this present disclosure can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glyceryl monostearate, talc, sodium chloride, dry skim milk, glycerol, propylene, glycols, water, ethanol, and the like. For the use of excipients and their uses, see also (“Handbook of Pharmaceutical Excipients”, Fifth Edition, R. C. Rowe, P. J. Seskey and S. C. Owen, Pharmaceutical Press, London, Chicago). The composition can also comprise minor amounts of wetting or emulsifying agents, or pH buffering agents. These compositions may take the form of solutions, suspensions, emulsions, tablets, pills, capsules, powders, sustained release formulations and the like. Oral formulations can comprise standard pharmaceutical carriers and/or excipients, such as pharmaceutical grades of mannitol, lactose, starch, magnesium stearate, saccharine.

In an embodiment, the present disclosure provides a method for preventing a coronavirus-related disease or condition in a subject, which comprises administering the aforementioned pharmaceutical composition to the subject.

Subjects at risk for a coronavirus-related disease comprises those who have been in contact with an infected person or who have been exposed to a coronavirus in some other way. Administration of preventive measures, such as vaccines, can be administered prior to the onset of symptoms relating to a coronavirus-associated disease. This helps prevent the disease from developing or slows down its progression.

In an embodiment, the present disclosure also provides a method of treating a coronavirus-associated disease in a patient. In an embodiment, the method involves administering an antibody of the present disclosure, or a pharmaceutical composition that neutralizes a coronavirus, to a patient suffering from said disease.

6. The antibody or its antigen-binding fragment of the present disclosure and the composition thereof is used for diagnosing and detecting coronavirus.

The antibodies or antigen-binding fragments in the present disclosure can be used to detect the presence of coronaviruses in biological samples, and then to diagnose and detect coronaviruses.

The anti-SARS-CoV-2 S protein antibody or antigen-binding fragment provided by the present disclosure can be conveniently used in a kit, and the SARS-CoV-2 S protein in biological fluids or tissues in vivo or in vitro can be detected by the antibody or antigen-binding fragment provided by the present disclosure. The kit can be used for the detection of any sample containing detectable amount of SARS-CoV-2 S protein.

The kits in the present disclosure may comprise at least one antibody or antigen-binding fragment. The kits may comprise one or more of the compositions in the present disclosure, optionally together with one or more other prophylactic or therapeutic agents for the diagnosis, prevention, control or treatment of SARS-CoV-2. The kit can also include instructions for preventing, treating, controlling or improving SARS-CoV-2, as well as side effects and dosage information of administration methods.

The presence of antibodies to the novel coronavirus in a test sample can indicate that the subject in which the sample was located is or has previously been infected with the new coronavirus. The detection of the presence of antibodies to the 2019-nCoV in a sample can indicate the immune response, especially the humoral immune response, of the subject before the sample to current or previous 2019-nCoV infection.

The samples in the present disclosure may include, but are not limited to, liquids such as urine, saliva, cerebrospinal fluid, blood, serum, and the like, or samples may be solid or semi-solid such as tissue, feces, and the like, or can be solid tissues such as those commonly used for histological diagnosis.

In some embodiments, the subject can be a patient with COVID-19, a patient who has recovered from COVID-19, or an individual who has been vaccinated against COVID-19.

The term “detection” as used in the present disclosure comprises quantitative or qualitative detection, and exemplary detection methods may involve immunohistochemistry, immunocytochemistry, flow cytometry (e.g., FACS), magnetic beads complexed with antibody molecules, and ELISA assays. In some embodiments, the anti-SARS-CoV-2 S protein antibody or antigen-binding fragment of the present disclosure can be coupled with detectable labels such as luciferase and biotinidase. These labeled antibodies can be utilized in both liquid and solid-phase immunoassays, including direct or indirect techniques like FACS, IHC, ELISA, whether competitive or non-competitive in nature.

In an embodiment, a method of detecting the presence of a coronavirus in a biological sample is provided. In some embodiments, the method may comprise the detection of whether coronavirus S protein exists in biological samples. In certain embodiments, the method comprises contacting a biological sample with a anti-coronavirus S protein antibody or antigen-binding fragment under conditions that allow the anti-coronavirus S protein antibody or antigen-binding fragment to bind to the coronavirus S protein, and detecting whether a complex is formed between the anti-coronavirus S protein antibody or antigen-binding fragment and the coronavirus S protein. The formation of complexes indicates the presence of coronavirus. The method can be an in vitro or in vivo method.

Exemplary diagnostic assays for coronaviruses comprise, for example, contacting a sample obtained from a patient with the anti-coronavirus S protein of the present disclosure, wherein the anti-coronavirus S protein is tagged with a detectable label (or marker) or reporter molecule or used as a capture ligand to selectively isolate the coronavirus from the patient sample. Alternatively, unlabeled anti-coronavirus S protein can be used in diagnostic applications in combination with a secondary antibody that is detectably labeled. The detectable label or reporter molecule can be a radioactive isotope such as 3H, 14C, 32P, 35S or 1251.

Fluorescent or chemiluminescent moieties such as fluorescein isothiocyanate or rhodamine, or enzymes such as alkaline phosphatase, beta-galactosidase, horseradish peroxidase, and luciferase. Specific exemplary assays that can be used to detect or measure coronavirus in a sample comprise enzyme-linked immunosorbent assay (ELISA), radioimmunoassay (RIA), and fluorescence-activated cell sorting (FACS).

Samples that can be used in a coronavirus diagnostic assay according to the present disclosure comprise any biological sample obtained from a patient that contains a detectable amount of the coronavirus spike protein or fragment under normal or physiological conditions. In some embodiments, the biological sample is blood, serum, throat swab, lower respiratory tract sample (e.g., tracheal secretions, tracheal aspirate, alveolar lavage fluid), and other sample of biological origin. In general, coronavirus spike protein levels may be measured in specific samples obtained from healthy patients (e.g., patients not disturbed by a coronavirus-associated disease) to initially establish baseline or standard coronavirus levels.

This baseline level of coronavirus can then be compared to the level of coronavirus measured in a sample obtained from an individual suspected of having a coronavirus-related condition or symptoms.

6. Other applications of the antibody or the antigen-binding fragment of the present disclosure.

It can be understood that based on the basic properties of the neutralizing antibody of the present disclosure, the antibody or antigen-binding fragment of the present disclosure can also be applied to the detection/screening/purification/preparation of neutralizing antibodies, etc.

The technical solutions of the present disclosure will be clearly and completely described below in conjunction with the embodiments. Apparently, the described embodiments are part of the embodiments of the present disclosure, not all of them. Based on the embodiments in this present disclosure, all other embodiments obtained by persons of ordinary skill in the art without making creative efforts belong to the scope of protection of this present disclosure.

### EXAMPLES

The technical solutions of the embodiments of the present disclosure will be more clearly described below, and the accompanying drawings that need to be configured in the description of the embodiments will be briefly described below.

Obviously, the drawings described below are only some examples or embodiments of the present disclosure. Those skilled in the art, without further creative efforts, may apply the present disclosure to other similar scenarios according to these drawings. Unless obviously obtained from the context or the context illustrates otherwise, the same numeral in the drawings refers to the same structure or operation.

### Example 1. Single Memory B Cell Sorting and Antibody Cloning

Volunteer recruitment and blood draw were approved by the ethics committee of School of Basic Medicine, Fudan University. Participants, four donors who received three doses of the inactivated CoronaVac vaccine and three donors who received two doses, donated blood one month after vaccination. All donors were between the ages of 23-52 with a male to female ratio of 3:4. After peripheral blood collection, human peripheral blood mononuclear cells (PBMCs) were isolated, aliquoted, and stored in liquid nitrogen.

Stored PBMC were thawed and incubated with CD19 microspheres (Miltenyi Biotec). CD19+B lymphocytes were then incubated sequentially with human Fc fragment (BD Biosciences), anti-CD20-PECy7 (BD Biosciences), S-ECD-PE and S-ECD-APC. Single memory B cells (CD20-PECy7+S-ECD-PE+S-ECD-APC+) were then sorted into 96-well plates using a FACSAria II (BD Biosciences) and used for antibody cloning. The amplified PCR products of immunoglobulin heavy chain and K/A light chain Fab regions were subjected to electrophoresis and Sanger sequencing. Their nucleotide sequences were analyzed by IMGT/V-QUEST and IgBlast, and the V(D)J gene fragment and CDR3 sequence of each antibody were determined.

### Example 2. Antibody Expression

Vector construction and antibody expression were performed on selected antibodies. Briefly, all cloned human monoclonal antibodies were prepared by transient transfection of mammalian HEK293F cells cultured in serum-free OPM-293-CD05 medium (OPM Biosciences) at 37° C., 5% CO₂, and shaking at 100 rpm.

A total of 30 cloned antibodies were expressed.

### Example 3. Pseudovirus Neutralization Experiment

In vitro neutralization assays were performed using pseudoviruses, as previously described. Huh-7 cells were seeded and then incubated with pseudovirus/antibody mixture for 12 hours. Antibodies were serially diluted 1:3 in PBS for a total of 9 dilutions starting at 10 μg/ml. Using fresh DMEM medium instead of mixture for further culturing. After 24 or 48 h, Huh-7 cells were collected and luminescence was measured as previously described. The experimental results are shown in Table 1.

TABLE 1
virus and the experimental results
pseudovirus IC50(μg/ml)

| Antibody | | B.1.1.7 | B.1.351 | P.1 | B.1.617.2 | C.37 | B.1.1.529 |
|---|---|---|---|---|---|---|---|
| Number | WT | (Alpha) | (Beta) | (Gamma) | (Delta) | (Lambda) | (Omicron) |
| ACRO3- | 0.020 | 0.009 | <0.01 | 0.007 | 0.008 | 0.017 | 0.006 |
| 286L | | | | | | | |
| ACRO3- | 0.068 | 0.024 | 0.009 | 0.010 | 0.016 | 0.024 | 0.006 |
| 347K | | | | | | | |
| ACRO2- | Neg | Neg | Neg | Neg | Neg | Neg | Neg |
| 001K | | | | | | | |
| ACRO2- | Neg | Neg | Neg | Neg | Neg | Neg | Neg |
| 021L | | | | | | | |
| ACRO2- | Neg | Neg | Neg | Neg | Neg | Neg | Neg |
| 028K | | | | | | | |
| ACRO3- | Neg | Neg | Neg | Neg | Neg | Neg | Neg |
| 168L | | | | | | | |
| ACRO3- | Neg | Neg | Neg | Neg | Neg | Neg | Neg |
| 169L | | | | | | | |
| ACRO3- | Neg | Neg | Neg | Neg | Neg | Neg | Neg |
| 207L | | | | | | | |
| ACRO3- | Neg | Neg | Neg | Neg | Neg | Neg | Neg |
| 283L | | | | | | | |
| ACRO3- | Neg | Neg | Neg | Neg | Neg | Neg | Neg |
| 307K | | | | | | | |
| ACRO3- | Neg | Neg | Neg | Neg | Neg | Neg | Neg |
| 309K | | | | | | | |
| ACRO3- | Neg | Neg | Neg | Neg | Neg | Neg | Neg |
| 341L | | | | | | | |
| ACRO3- | Neg | Neg | Neg | Neg | Neg | Neg | Neg |
| 410K | | | | | | | |
| ACRO3- | Neg | Neg | Neg | Neg | Neg | Neg | Neg |
| 411L | | | | | | | |
| ACRO3- | Neg | Neg | Neg | Neg | Neg | Neg | Neg |
| 414K | | | | | | | |

Note:
Neg refers to negative.

It can be seen from Table 1 that the antibodies ACRO3-286L and ACR03-347K have better neutralizing ability against pseudovirus strains WT, B.1 0.1 0.7 (Alpha), B.1.351 (Beta), P.1 (Gamma), B.1 0.61 7.2 (Delta), 0.37 (Lambda) and B.1.1.529 (Omicron).

### Example 4. Authentic Virus Neutralization Experiment

Plasma samples collected from convalescent and vaccinated volunteers were first inactivated at 56 00 for 0.5 h. Inactivated serum samples or purified mAbs were serially diluted with cell culture medium from 1:4 or 50,000 ng/mL in two steps and mixed with virus suspension containing 100 TCID50 and incubated at 36.50C for 2 hours. Then, the mixture was added to a 96-well plate seeded with confluent Vero cells and incubated for another 5 days in an incubator at 36.5 00 5% CO₂. The cytopathic effect (CPE) of each well was observed and recorded microscopically by three different individuals, and then used to calculate neutralization titers by the Reed-Muench method. The experimental results are shown in Table 2.

TABLE 2
Results of authentic virus neutralization
experiment authentic virus IC50(μg/ml)

| Antibody | | B.1.351 | P.1 | B.1.617.2 |
|---|---|---|---|---|
| Number | WT | (Beta) | (Gamma) | (Delta) |
| ACRO3-286L | 0.02 | 0.02 | 0.02 | 0.02 |
| ACRO3-347K | 0.02 | 0.02 | 0.024 | 0.033 |
| ACRO2-001K | 4.167 | 12.500 | 16.667 | 25.000 |
| ACRO2-021L | 6.250 | 50 | 33.333 | 50.000 |
| ACRO2-028K | 6.250 | 50 | 50.000 | 50 |
| ACRO3-168L | 3.125 | 50.000 | 16.667 | 50 |
| ACRO3-169L | 3.125 | 16.667 | 50.000 | 50 |
| ACRO3-207L | 3.125 | 50 | 50 | 50 |
| ACRO3-283L | 0.033 | 50 | 50.000 | 50 |
| ACRO3-307K | 3.125 | 25.000 | 33.333 | 16.667 |
| ACRO3-309K | 3.125 | 12.500 | 16.667 | 12.500 |
| ACRO3-341L | 6.250 | 50 | 50 | 50 |
| ACRO3-410K | 4.167 | 25.000 | 50 | 8.333 |
| ACRO3-411L | 0.098 | 50 | 25.000 | 50 |
| ACRO3-414K | 0.033 | 50 | 33.333 | 50 |

As shown in Table 2, in terms of authentic virus neutralization, the antibodies ACRO3-286L and ACRO3-347K displayed higher neutralizing titers in WT, B.1 0.351 (Beta), P.1 (Gamma), and B.1 0.617.2 (Delta), indicating these antibodies have the high neutralization activity.

The above descriptions of specific exemplary embodiments of the present disclosure have been presented for purposes of illustration and description. These descriptions are not intended to limit the present disclosure to the precise form disclosed, and obviously many modifications and variations are possible in light of the above teaching. The exemplary embodiments were chosen and described in order to explain certain principles of the present disclosure and their practical present disclosure, thereby enabling those skilled in the art to make and utilize various exemplary embodiments and various alternatives and modifications of the present disclosure. It is intended that the scope of the present disclosure be defined by the claims and their equivalents.

1. An antibody or antigen-binding fragment against SARS-CoV-2 spike(S) protein, comprising:
three complementarity determining regions (HCDRs) of a heavy chain variable region or one or more variants thereof, the heavy chain variable region set forth as SEQ ID NO. 30 or SEQ ID NO. 46, each of the one or more variants having at most two amino acid changes compared to the corresponding CDR; and
three complementarity determining regions (LCDRs) of a light chain variable region or one or more variants thereof, the light chain variable region set forth as SEQ ID NO. 32 or SEQ ID NO. 48, each of the one or more variants having at most two amino acid changes compared to the corresponding CDR.

2. The antibody or the antigen-binding fragment of claim 1, wherein the HCDRs and LCDRs are numbered by a Kabat numbering scheme, and the antibody or the antigen-binding fragments includes:
i. HCDR1 set forth as SEQ ID NO. 103 or 127 or a variant thereof, wherein the variant has at most two amino acid changes compared to the HCDR1;
ii. HCDR2 set forth as SEQ ID NO. 104 or 128 or a variant thereof, wherein the variant has at most two amino acid changes compared to the HCDR2;
iii. HCDR3 set forth as SEQ ID NO. 105 or 129 or a variant thereof, wherein the variant has at most two amino acid changes compared to the HCDR3;
iv. LCDR1 set forth as SEQ ID NO. 106 or 130 or a variant thereof, wherein the variant has at most two amino acid changes compared to the LCDR1;
v. LCDR2 set forth as SEQ ID NO. 107 or 131 or a variant thereof, wherein the variant has at most two amino acid changes compared to the LCDR2;
vi. LCDR3 set forth as SEQ ID NO. 108 or 132 or a variant thereof, wherein the variant has at most two amino acid changes compared to the LCDR3.

3. The antibody or the antigen-binding fragment of claim 2, wherein
(i) the HCDR1, the HCDR2, and the HCDR3 are selected from a group set forth as: SEQ ID NO. 103, SEQ ID NO. 104, and SEQ ID NO. 105, respectively; or SEQ ID NO. 127, SEQ ID NO. 128, and SEQ ID NO. 129, respectively; and
(ii) each of the variants of the HCDR1, HCDR2, and HCDR3 has at most two amino acid substitutions, deletions or insertions compared to the corresponding HCDR1, HCDR2, or HCDR3 in (i);
(iii) LCDR1, LCDR2, and LCDR3 are selected from a group set forth as: SEQ ID NO. 106, SEQ ID NO. 107, and SEQ ID NO. 108, respectively; or SEQ ID NO. 130, SEQ ID NO. 131, and SEQ ID NO. 132, respectively; and
(iv) each of the variants of the LCDR1, LCDR2, and LCDR3 has at most two amino acid substitutions, deletions or insertions compared to the corresponding LCDR1, LCDR2, or LCDR3 in (iii).

4. The antibody or the antigen-binding fragment of claim 1, wherein the antibody or the antigen-binding fragment includes
a) the heavy chain variable region having an amino acid sequence set forth as SEQ ID NO. 30 or SEQ ID NO. 46, or that is at least 70%, 80%, 90%, 95%, 99% or 100% similarity with SEQ ID NO. 30 or SEQ ID NO. 46; and
b) the light chain variable region having an amino acid sequence set forth as SEQ ID NO. 32 or SEQ ID NO. 48, or that is at least 70%, 80%, 90%, 95%, 99% or 100% similarity with SEQ ID NO. 32 or SEQ ID NO. 48.

5. The antibody or antigen-binding fragment of claim 1, wherein the antibody or antigen-binding fragment further comprises a coupling moiety connected to a polypeptide, and the coupling moiety is selected from at least one of radionuclides, drugs, toxins, cytokines, enzymes, fluoresceins, carrier proteins, lipids, and biotin, wherein the polypeptide or the antibody is selectively linked to the coupling moiety by a linker, the linker is a peptide or a polypeptide.

6. The antibody or antigen-binding fragment of claim 1, wherein the antibody or antigen-binding fragment is selected from monoclonal antibodies, polyclonal antibodies, antiserum, chimeric antibodies, humanized antibodies, and human antibodies.

7. An isolated polynucleotide, wherein the polynucleotide encodes the antibody or antigen-binding fragment of claim 1.

8. A recombinant vector comprising the polynucleotide of claim 7, and an optional regulatory sequence; wherein
the recombinant vector is a cloning vector or an expression vector;
the regulatory sequence is selected from a leader sequence, a polyadenylation sequence, a propeptide sequence, a promoter, a signal sequence, a transcription terminator, or any combination thereof.

9. A host cell, comprising the recombinant vector of claim 8; wherein
the host cell is a prokaryotic cell or a eukaryotic cell.

10. A pharmaceutical composition, comprising any one or more of the antibody or antigen-binding fragment of claim 1, a polynucleotide encoding the antibody or antiqen-bindinq fragment, a recombinant vector comprising the antibody or antiqen-bindinq fragment, or a host cell comprising the recombinant vector; and a pharmaceutically acceptable carrier or adjuvant.

11. A kit, comprising one or more of the antibody or antigen-binding fragment, polynucleotide, recombinant vector, and host cell of claim 1, wherein the kit is contained in a suitable container.

12. (canceled)

13. A method for preparing a neutralizing antibody or an antigen-binding fragment capable of binding to SARS-CoV-2 virus, comprising
expressing the recombinant vector in a culture containing the host cell of claim 9 to produce the antibody; and
recovering the antibody from the culture.

14. A method for preventing or treating COVID-19, comprising
administering to a subject an effective amount of the antibody or antigen-binding fragment of claim 1, or the pharmaceutical composition that comprises the antibody or antigen-binding fragment.

15. The antibody or the antigen-binding fragment of claim 6, wherein the antibody is selected from multispecific antibodies, single-chain Fv (scFv), single-chain antibodies, anti-idiotype (anti-Id) antibodies, diabodies, minibodies, nanobodies, single domain antibodies, Fab fragments, F(ab′) fragments, disulfide-linked bispecific Fv (sdFv) and intrabodies

16. The host cell of claim 9, wherein the host cell includes a yeast cell, a Chinese hamster ovary cell, or a human embryonic kidney cell.


SEQUENCE LISTING (150 sequences)

SEQ ID NO: 1 - DNA, 363 bp, synthetic construct
  misc_feature 1..363: note = ACRO2-001K
  source 1..363: mol_type = other DNA
gaggtgcagctggtggagtctggaggaggcgtgatccagcctggggggtccctgagactctcctgtgcagcctctgggttcaccgtcagtgccaactacatgagctgggtccgccaggctccagggaaggggctggagtgggtctcagttatttatagcggtggcagcacaaacgatggtgacacatactacgcagcctccgtgaagggccgattcaccatctccagagacaattccaagaacacgctgtatcttcaaatgaacagcctgagagccgaggacacggccgtgtattactgtgcgagagtggtgtctgatgcttttgatatctggggccaaggggcaatggtcaccgtgtcttct

SEQ ID NO: 2 - protein, 121 aa, synthetic construct
  REGION 1..121: note = ACRO2-001K
EVQLVESGGGVIQPGGSLRLSCAASGFTVSANYMSWVRQAPGKGLEWVSVIYSGGSTNDGDTYYAASVKGRFTISRDNSKNTLYLQMNSLRAEDTAVYYCARVVSDAFDIWGQGAMVTVSS

SEQ ID NO: 3 - DNA, 321 bp, synthetic construct
  misc_feature 1..321: note = ACRO2-001K
  source 1..321: mol_type = other DNA
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctataaggcatctattttagaaagtggggtcccatcaaggttcagcggcagtggatctgggacagaattcactctcaccatcagcagcctgcagcctgatgattttgcaacttattactgccaacactataatagcgattcctacacttttggccaggggaccaagctggagatcaaa

SEQ ID NO: 4 - protein, 107 aa, synthetic construct
  REGION 1..107: note = ACRO2-001K
DIQMTQSPSTLSASVGDRVTITCRASQSISSWLAWYQQKPGKAPKLLIYKASILESGVPSRFSGSGSGTEFTLTISSLQPDDFATYYCQHYNSDSYTFGQGTKLEIK

SEQ ID NO: 5 - DNA, 363 bp, synthetic construct
  misc_feature 1..363: note = ACRO2-021L
  source 1..363: mol_type = other DNA
gaggtgcagctgttggagtctgggggaggcttggtacagcctggggggtccctgagactctcctgtgcagcctctggattcacctttagcagctttgccatgacctgggtccgccaggctccagggaaggggctggagtgcgtctcagttattagtggtggtgctggtagcacatactacgcagactccgtgaagggccggttcaccctctccagagacaattccaagaacacgctgtatctgcaaatgaacagcctgagagccgaggacacggccgtatattactgtgcgaaaggggagaggcccgactacggtgactactttgactactggggccagggaaccctggtcaccgtctcctca

SEQ ID NO: 6 - protein, 121 aa, synthetic construct
  REGION 1..121: note = ACRO2-021L
EVQLLESGGGLVQPGGSLRLSCAASGFTFSSFAMTWVRQAPGKGLECVSVISGGAGSTYYADSVKGRFTLSRDNSKNTLYLQMNSLRAEDTAVYYCAKGERPDYGDYFDYWGQGTLVTVSS

SEQ ID NO: 7 - DNA, 327 bp, synthetic construct
  misc_feature 1..327: note = ACRO2-021L
  source 1..327: mol_type = other DNA
tcttatgtgctgactcagcccccctcagtgtcagtggccccaggaaagacggccaggattacctgtgggggaaacaacattggaagtaaaagtgtgcactggtaccagcagaggccaggccaggcccctgtcctggtcatgtattatgatagcgaccggccctcagggatccctgaccgattctctggctccaactctgggaacacggccaccctgaccatcagccgggtcgaagccggggatgaggccgactattactgtcaggtgtgggatggtagtagtgatcatcattatgtcttcggcactgggaccaagctgacagtgctg

SEQ ID NO: 8 - protein, 109 aa, synthetic construct
  REGION 1..109: note = ACRO2-021L
SYVLTQPPSVSVAPGKTARITCGGNNIGSKSVHWYQQRPGQAPVLVMYYDSDRPSGIPDRFSGSNSGNTATLTISRVEAGDEADYYCQVWDGSSDHHYVFGTGTKLTVL

SEQ ID NO: 9 - DNA, 348 bp, synthetic construct
  misc_feature 1..348: note = ACRO2-028K
  source 1..348: mol_type = other DNA
gaggtgcagctggtggagtctggaggaggcttggtccagcctggggggtccctgagactctcctgtgcagcctctgggttcagcgtcagtagcaactacatgagctgggtccgccaggctccagggaaggggctggagtgggtctcagttatttatagcggtggtagcacatactacgcagactccgtgaagggccgattcaccatctccagacacaattccaagaacacgctgtatcttcaaatgaacagcctgagagctgaggacacggccgtgtattactgtgcgagagtcgtaaccgacgcttttgatatctggggccaagggacaatggtcaccgtctcttca

SEQ ID NO: 10 - protein, 116 aa, synthetic construct
  REGION 1..116: note = ACRO2-028K
EVQLVESGGGLVQPGGSLRLSCAASGFSVSSNYMSWVRQAPGKGLEWVSVIYSGGSTYYADSVKGRFTISRHNSKNTLYLQMNSLRAEDTAVYYCARVVTDAFDIWGQGTMVTVSS

SEQ ID NO: 11 - DNA, 321 bp, synthetic construct
  misc_feature 1..321: note = ACRO2-028K
  source 1..321: mol_type = other DNA
gacatccagatgacccagtctccttccaccctgtctgcatctgttggagacagagtcaccatcacttgccgggccagtcagagtattagtagctggttggcctggtatcagcagacaccagggaaagcccctaagctcctgatctataaggcatctagtttagaaagtggggtcccatcaaggttcagcggcagtggatctgggacagaattcactctcaccatcagcagcctgcagcctgatgattttgcaatttattactgccaacagtataatagtgattcgtacacttttggccaggggaccaagctggagatcaaa

SEQ ID NO: 12 - protein, 107 aa, synthetic construct
  REGION 1..107: note = ACRO2-028K
DIQMTQSPSTLSASVGDRVTITCRASQSISSWLAWYQQTPGKAPKLLIYKASSLESGVPSRFSGSGSGTEFTLTISSLQPDDFAIYYCQQYNSDSYTFGQGTKLEIK

SEQ ID NO: 13 - DNA, 360 bp, synthetic construct
  misc_feature 1..360: note = ACRO3-168L
  source 1..360: mol_type = other DNA
cagctgcagctgcaggagtcgggcccaggactggtgaagccttcggagaccctgtccctcacctgcactgtctctggtggctccatcagcagtagtcgttactactggggctggatccgccagcccccagggaaggggctggagtggattgggagtctctattatagtgggggcacctacttcaacccgtccctcaagagtcgagtcaccatctcagtagacacgtccaagaaccagttctccctgaaactgacctctgtgaccgccgcggacacggccgtttattactgtgcgagagaccattacggtgactacgcccttgtctactggggccagggaaccctggtcaccgtctcctca

SEQ ID NO: 14 - protein, 120 aa, synthetic construct
  REGION 1..120: note = ACRO3-168L
QLQLQESGPGLVKPSETLSLTCTVSGGSISSSRYYWGWIRQPPGKGLEWIGSLYYSGGTYFNPSLKSRVTISVDTSKNQFSLKLTSVTAADTAVYYCARDHYGDYALVYWGQGTLVTVSS

SEQ ID NO: 15 - DNA, 336 bp, synthetic construct
  misc_feature 1..336: note = ACRO3-168L
  source 1..336: mol_type = other DNA
cagtctgtgctgacgcagccgccctcagtgtctggggccccagggcagagggtcaccatctcctgcactgggagcagctccaacatcggggcaggttatgatgtacactggtaccagcagcttccaggaacagcccccaaactcctcatctatggtcacaacaatcggccctcaggggtccctgaccgattctctggctcccagtctggcacctcagcctccctggccatcactgggctccaggctgaggatgaggctgattattactgccagtcctatgacactagcctgagtgccctttatgtcttcggaactgggaccaaggtcaccgtcctg

SEQ ID NO: 16 - protein, 112 aa, synthetic construct
  REGION 1..112: note = ACRO3-168L
QSVLTQPPSVSGAPGQRVTISCTGSSSNIGAGYDVHWYQQLPGTAPKLLIYGHNNRPSGVPDRFSGSQSGTSASLAITGLQAEDEADYYCQSYDTSLSALYVFGTGTKVTVL

SEQ ID NO: 17 - DNA, 360 bp, synthetic construct
  misc_feature 1..360: note = ACRO3-169L
  source 1..360: mol_type = other DNA
cagctgcagctgcaggagtcgggcccaggactggtgaagccttcggagaccctgtccctcacctgcactgtctctggtggctccatcagcagtagtcgttactactggggctggatccgccagcccccagggaaggggctggagtggattgggagtctgtattatagtgggggcacctactacaacccgtccctcaagagtcgagtcaccatctcagtagacacgtccaagaaccagttctccctgaaactgacctctgtgaccgccgcggacacggccgtttattactgtgcgagagaccattacggtgactacgcccttgtctactggggccagggaaccccagtcaccgtctcctca

SEQ ID NO: 18 - protein, 120 aa, synthetic construct
  REGION 1..120: note = ACRO3-169L
QLQLQESGPGLVKPSETLSLTCTVSGGSISSSRYYWGWIRQPPGKGLEWIGSLYYSGGTYYNPSLKSRVTISVDTSKNQFSLKLTSVTAADTAVYYCARDHYGDYALVYWGQGTPVTVSS

SEQ ID NO: 19 - DNA, 336 bp, synthetic construct
  misc_feature 1..336: note = ACRO3-169L
  source 1..336: mol_type = other DNA
cagtctgtgctgacgcagccgccctcagtgtctggggccccagggcagagggtcaccatctcctgcactgggagcagctccaacgtcggggcaggttatgatgtacactggtaccagcagcttccaggaacagcccccaaactcctcatctatggtaacaacaatcggccctcaggggtccctgaccgattctctggctcccagtctggcacctcagcctccctggccatcactgggctccagactgaggatgaggctgattattactgccagtcctatgacactagcctgagtgccctttatgtcttcggaactgggaccaaggtcaccgtgctg

SEQ ID NO: 20 - protein, 112 aa, synthetic construct
  REGION 1..112: note = ACRO3-169L
QSVLTQPPSVSGAPGQRVTISCTGSSSNVGAGYDVHWYQQLPGTAPKLLIYGNNNRPSGVPDRFSGSQSGTSASLAITGLQTEDEADYYCQSYDTSLSALYVFGTGTKVTVL

SEQ ID NO: 21 - DNA, 354 bp, synthetic construct
  misc_feature 1..354: note = ACRO3-207L
  source 1..354: mol_type = other DNA
caggtgcagctggtgcaatctgggtctgagttgaagaagcctggggcctcagtgaaggtttcctgcaaggcctctggatacagcttcactagttatgctatgaattgggtgcgacaggcccctggacaagggcttgagtggatgggatggatcaataccaatactgggaacccaacgtatgcccagggcttcacaggacggtttgtcttctccttggacacctctgtcaacacggcatatctgcagatcagcggcctgaaggctgaggacactgccgtctattactgtgcgagagagaccagttgggatggctatatctattggggccagggaaccctggtcaccgtctcctca

SEQ ID NO: 22 - protein, 118 aa, synthetic construct
  REGION 1..118: note = ACRO3-207L
QVQLVQSGSELKKPGASVKVSCKASGYSFTSYAMNWVRQAPGQGLEWMGWINTNTGNPTYAQGFTGRFVFSLDTSVNTAYLQISGLKAEDTAVYYCARETSWDGYIYWGQGTLVTVSS

SEQ ID NO: 23 - DNA, 324 bp, synthetic construct
  misc_feature 1..324: note = ACRO3-207L
  source 1..324: mol_type = other DNA
tcctatgaggtgacacagccaccctcggtgtcagtgtccccaggacagacggccaggatcacctgctctggagatacattgtcaaagcaatatactttttggtaccaacagaagacaggccaggcccctgtgttggtgatatataaagacagtgagaggccctcagggatccctgagcgattctctggctccagctcagggacaacagtcaccttgaccatcagtggagtccaggcagaagacgaggctgactattattgtcaatcagcagacaacagtggtacttcttgggtgttcggcggagggaccaagctgacagtgctg

SEQ ID NO: 24 - protein, 108 aa, synthetic construct
  REGION 1..108: note = ACRO3-207L
SYEVTQPPSVSVSPGQTARITCSGDTLSKQYTFWYQQKTGQAPVLVIYKDSERPSGIPERFSGSSSGTTVTLTISGVQAEDEADYYCQSADNSGTSWVFGGGTKLTVL

SEQ ID NO: 25 - DNA, 369 bp, synthetic construct
  misc_feature 1..369: note = ACRO3-283L
  source 1..369: mol_type = other DNA
caggtccagctggtacagtctggggctgaggtgaagaagcctggggcctcagtgaaggtctcctgcaaggtttccggatacaccctcattgaattatccatgcactgggtgcgacaggctcctggaaaagggcttgagtggatgggaggttttgatcctgaagatgctgaaacaatctacgcacagaagttccagggcagagtcaccatgaccgaggacacatctacagacacagcctatatggagctgagcaggctgagatctgaggacacggccgtgtattactgtgcaataggtcccgcgataccaatggctgctactgggtggttcgacccctggggccagggaaccctggtcaccgtctcctca

SEQ ID NO: 26 - protein, 123 aa, synthetic construct
  REGION 1..123: note = ACRO3-283L
QVQLVQSGAEVKKPGASVKVSCKVSGYTLIELSMHWVRQAPGKGLEWMGGFDPEDAETIYAQKFQGRVTMTEDTSTDTAYMELSRLRSEDTAVYYCAIGPAIPMAATGWFDPWGQGTLVTVSS

SEQ ID NO: 27 - DNA, 330 bp, synthetic construct
  misc_feature 1..330: note = ACRO3-283L
  source 1..330: mol_type = other DNA
cagtctgccctgactcagcctgcctccgtgtctgggtctcctggacagtcgatcaccatctcctgcactggaaccagcagtgacgttggtggttataactatgtctcctggtaccaacagcacccaggcaaagcccccaaactcatgatttatgaggtcagtaatcggccctcaggggtttctaatcgcttctctggctccaagtctggcaacacggcctccctgaccatctctgggctccaggctgaggacgaggctgattattactgcagctcatatacaagcagcagcacttatgtcttcggaactgggaccaaggtcaccgtgctg

SEQ ID NO: 28 - protein, 110 aa, synthetic construct
  REGION 1..110: note = ACRO3-283L
QSALTQPASVSGSPGQSITISCTGTSSDVGGYNYVSWYQQHPGKAPKLMIYEVSNRPSGVSNRFSGSKSGNTASLTISGLQAEDEADYYCSSYTSSSTYVFGTGTKVTVL

SEQ ID NO: 29 - DNA, 363 bp, synthetic construct
  misc_feature 1..363: note = ACRO3-286L
  source 1..363: mol_type = other DNA
caggtgcagctggtgcagtctggggctgaggtgaagaagcctggggcctcagtgaaggtttcctgcaaggcatctggatacaccttctccagctactatatacactgggtgcgacaggcccctggacaagggcctgagtggatggcaataatcaaccctggtgatggtggtgcaagttacgcacagaaatttcagggcagagtcaccctgaccagggacacgtccacgagcacactttacatggagctgagcagcctgagatctgaggatacggccgtgtattactgtgcgagagccgagggcagcagctggcttgggtggttcgacccctggggccagggaaccctggtcaccgtctcctca

SEQ ID NO: 30 - protein, 121 aa, synthetic construct
  REGION 1..121: note = ACRO3-286L
QVQLVQSGAEVKKPGASVKVSCKASGYTFSSYYIHWVRQAPGQGPEWMAIINPGDGGASYAQKFQGRVTLTRDTSTSTLYMELSSLRSEDTAVYYCARAEGSSWLGWFDPWGQGTLVTVSS

SEQ ID NO: 31 - DNA, 336 bp, synthetic construct
  misc_feature 1..336: note = ACRO3-286L
  source 1..336: mol_type = other DNA
cagtctgtgctgactcagccaccctcagcgtctgggacccccgggcagagggtcaccatctcttgttctggaagcagctccaacatcggaagtaattatgtatactggtaccagcagctcccaggaacggcccccaaactcctcatctataggaataatcagcggccctcaggggtccctgaccgattctctggctccaggtctggcacctcagcctccctggccatcagtgggctccggtccgaggatgaggctgattattactgtgcagcatgggatgacggcctgagtggttcggggtgggtgttcggcggagggaccaagctgaccgtgctg

SEQ ID NO: 32 - protein, 112 aa, synthetic construct
  REGION 1..112: note = ACRO3-286L
QSVLTQPPSASGTPGQRVTISCSGSSSNIGSNYVYWYQQLPGTAPKLLIYRNNQRPSGVPDRFSGSRSGTSASLAISGLRSEDEADYYCAAWDDGLSGSGWVFGGGTKLTVL

SEQ ID NO: 33 - DNA, 360 bp, synthetic construct
  misc_feature 1..360: note = ACRO3-307K
  source 1..360: mol_type = other DNA
caggtgcagctggtggagtctgggggaggcgtggtccagcctgggaggtccctgagactctcctgttcagcgtctggattcatcttcagtaattatgccatgcactgggtccgccaggccccaggcaaggggctggagtgggtggcagttatatgggatgatggaagtaataaacactatgcagactccgtgaagggccgattcaccatctccagagacaattccaagaacacgctttatctgcaaatgaacagcctgagagccgaggacacggctgtgtattactgtgcgagagatcaaggggagctagttacttactttgactactggggccagggaaccctggtcatcgtctcttca

SEQ ID NO: 34 - protein, 120 aa, synthetic construct
  REGION 1..120: note = ACRO3-307K
QVQLVESGGGVVQPGRSLRLSCSASGFIFSNYAMHWVRQAPGKGLEWVAVIWDDGSNKHYADSVKGRFTISRDNSKNTLYLQMNSLRAEDTAVYYCARDQGELVTYFDYWGQGTLVIVSS

SEQ ID NO: 35 - DNA, 324 bp, synthetic construct
  misc_feature 1..324: note = ACRO3-307K
  source 1..324: mol_type = other DNA
gacatccagatgacccagtctccatcctccctgtctgcatctgtaggagacagagtcaccatcacttgccgggcaagtcagaccattagcagctatttaaattggtatcagcagaaaccagggaaagcccctaaactcctgatctatactacatccagtttgcaaagtggggttccatcacggttcagtggcagtggatctgggacagatttcactctcaccatcagcagtctgcaacctgaagattttgcaacttactactgtcaacagagttacagtaccccttcgtggacgttcggccaagggaccaaggtggaaatcaaa

SEQ ID NO: 36 - protein, 108 aa, synthetic construct
  REGION 1..108: note = ACRO3-307K
DIQMTQSPSSLSASVGDRVTITCRASQTISSYLNWYQQKPGKAPKLLIYTTSSLQSGVPSRFSGSGSGTDFTLTISSLQPEDFATYYCQQSYSTPSWTFGQGTKVEIK

SEQ ID NO: 37 - DNA, 363 bp, synthetic construct
  misc_feature 1..363: note = ACRO3-309K
  source 1..363: mol_type = other DNA
gaggtgcagctggtggagtctgggggaggcttggtacagccgggggggtccctgagactctcctgtggagcctctggattcagcttcagtaactacgacatacactgggtccgccaagctataggaacaggtctggagtgggtctcatgtgttggtacttatggtgacacatactattcagactccgtgaagggccgattcaccatctccagagaaaatgccaagaactccttgtatcttcaaatgaacagcctgagactcggagacacggctgtatatttctgtgcaagaggctcggtagaccctacgaccaactggttcttcgatctctggggccgtggcaccctggtcactgtctcctca

SEQ ID NO: 38 - protein, 121 aa, synthetic construct
  REGION 1..121: note = ACRO3-309K
EVQLVESGGGLVQPGGSLRLSCGASGFSFSNYDIHWVRQAIGTGLEWVSCVGTYGDTYYSDSVKGRFTISRENAKNSLYLQMNSLRLGDTAVYFCARGSVDPTTNWFFDLWGRGTLVTVSS

SEQ ID NO: 39 - DNA, 324 bp, synthetic construct
  misc_feature 1..324: note = ACRO3-309K
  source 1..324: mol_type = other DNA
gacatccagatgacccagtctccatcctccctgtctgcatctgtaggagacagagtcaccatcacttgccgggcgagtcagggcattaggaattttttggcctggtatcagcagaaaccagggaaagcccctaaactcctactctctgctgcatccagattggaaagtggggtcccatcgaggttcagtggcagtggatctgggacggattacactctcaccatcagcagcctgcagcctgaagattttgcaacttattactgtcaacagtattatagttcccctccgatcaccttcgcccaagggacacgactggagattaaa

SEQ ID NO: 40 - protein, 108 aa, synthetic construct
  REGION 1..108: note = ACRO3-309K
DIQMTQSPSSLSASVGDRVTITCRASQGIRNFLAWYQQKPGKAPKLLLSAASRLESGVPSRFSGSGSGTDYTLTISSLQPEDFATYYCQQYYSSPPITFAQGTRLEIK

SEQ ID NO: 41 - DNA, 390 bp, synthetic construct
  misc_feature 1..390: note = ACRO3-341L
  source 1..390: mol_type = other DNA
gaggtgcagctggtggagtctgggggaggcttggtacagcctggggggtccctgagactctcctgtgcagcctctggattcaccttcagtagctatagcatgaactgggtccgccaggctccagggaaggggctggagtgggtttcatacattagtagtagtaatattaccatatactacgcagactctgtgaagggccgattcaccatctccagagacaatgccaagaactcactgtatatgcaagtgaacagcctgagagacgaggacacggctgtgtattactgtgcgagagtcctacggtattatgattacgtttgggggagttatcaacgggaactctacggtatggacgtctggggccaagggaccacggtcaccgtctcctca

SEQ ID NO: 42 - protein, 130 aa, synthetic construct
  REGION 1..130: note = ACRO3-341L
EVQLVESGGGLVQPGGSLRLSCAASGFTFSSYSMNWVRQAPGKGLEWVSYISSSNITIYYADSVKGRFTISRDNAKNSLYMQVNSLRDEDTAVYYCARVLRYYDYVWGSYQRELYGMDVWGQGTTVTVSS

SEQ ID NO: 43 - DNA, 336 bp, synthetic construct
  misc_feature 1..336: note = ACRO3-341L
  source 1..336: mol_type = other DNA
cagtctgccctgactcagcctgcctccgtgtctgggtctcctggacagtcgatcaccatctcctgcactggaaccagcagtgacgttggtggttatgactatgtctcctggtaccagcaacaccccggcaaagcccccaaactgatgatttatgatgtcagtaatcggccctcaggggtttctaatcgcttctctggctccaagtctggcaacacggcctccctgaccatctctgggctccaggctgaggacgaggctgattattactgcagctcatttacatccagcagcactctcccctttgtcatcggaactgggaccaaggtcaccgtccta

SEQ ID NO: 44 - protein, 112 aa, synthetic construct
  REGION 1..112: note = ACRO3-341L
QSALTQPASVSGSPGQSITISCTGTSSDVGGYDYVSWYQQHPGKAPKLMIYDVSNRPSGVSNRFSGSKSGNTASLTISGLQAEDEADYYCSSFTSSSTLPFVIGTGTKVTVL

SEQ ID NO: 45 - DNA, 372 bp, synthetic construct
  misc_feature 1..372: note = ACRO3-347K
  source 1..372: mol_type = other DNA
caaatgcagctggtgcagtctgggcctgaggtgaagaagcctgggacctcagtgaaggtctcctgcaaggcttctggattcacctttactgacgtctcttctctgcaatgggtgcgacaggctcgtggacaacgccttgagtggataggatggacggtcgttggcactggtaatacaaactacgcaccgaggttccaagaaagagtcaccattaccacggacaagtccacaagcacggcctacatggaactgagcagcctgagatccgaggacacggccgtgtattactgtgcggcaccgttttgtagtgagacttcctgtagtgatggctttgatctgtggggccaagggacaaaggtcaccgtctcttca

SEQ ID NO: 46 - protein, 124 aa, synthetic construct
  REGION 1..124: note = ACRO3-347K
QMQLVQSGPEVKKPGTSVKVSCKASGFTFTDVSSLQWVRQARGQRLEWIGWTVVGTGNTNYAPRFQERVTITTDKSTSTAYMELSSLRSEDTAVYYCAAPFCSETSCSDGFDLWGQGTKVTVSS

SEQ ID NO: 47 - DNA, 324 bp, synthetic construct
  misc_feature 1..324: note = ACRO3-347K
  source 1..324: mol_type = other DNA
gaaattgtgttgacgcagtctccaggcaccctgtctttgtctccaggggacagagccaccctctcatgcagggccagtcagagtgttaggatctcctacttagcctggtaccagcagaaacctggccaggctcccaggctcctcatctctggttcgtccagtagggccactggcatcccagacaggttcagtgccagtgggtctgggacagacttcgctctcaccatcagcagactggagcctgaagattttgcagtatattactgtcagcagtatgataactcaccgtggacgttcggccaagggaccaaagtggaagtcaaa

SEQ ID NO: 48 - protein, 108 aa, synthetic construct
  REGION 1..108: note = ACRO3-347K
EIVLTQSPGTLSLSPGDRATLSCRASQSVRISYLAWYQQKPGQAPRLLISGSSSRATGIPDRFSASGSGTDFALTISRLEPEDFAVYYCQQYDNSPWTFGQGTKVEVK

SEQ ID NO: 49 - DNA, 369 bp, synthetic construct
  misc_feature 1..369: note = ACRO3-410K
  source 1..369: mol_type = other DNA
caggttcagctggtgcagtctggggctgaggtgaagaagcctggggcctcagtgaaggtctcctgcaaggcttctggttacacctttaccaactacggtatcagctgggtgcgacaggcccctggacaagggcttgagtggatgggatggatcagcagttacaatggtaatacacactatgcacggaagctccagggcagagtcaccatgaccacagacacatccacgagcacagtctacatggagctgaggagcctgagatctgacgacacggccgtgtattactgtgcgagagaaaattttaactacggtgactacatcatttacttcgacccctggggccagggaaccctggtcaccgtctcctca

SEQ ID NO: 50 - protein, 123 aa, synthetic construct
  REGION 1..123: note = ACRO3-410K
QVQLVQSGAEVKKPGASVKVSCKASGYTFTNYGISWVRQAPGQGLEWMGWISSYNGNTHYARKLQGRVTMTTDTSTSTVYMELRSLRSDDTAVYYCARENFNYGDYIIYFDPWGQGTLVTVSS

SEQ ID NO: 51 - DNA, 321 bp, synthetic construct
  misc_feature 1..321: note = ACRO3-410K
  source 1..321: mol_type = other DNA
gaaattgtgctgactcagtctccagactttcagtctgtgactccaaaggagaaagtcaccatcacctgccgggccagtgaaagcattggtagtagcttacactggtaccagcagaaaccagatcagtctccaaaactcctcatcaagcatgcttcccagtccttctcaggggtcccctcgaggttcagtggcagtggatctgggacagatttcaccctcaccatcaatggcctggaagctgaagatgctgcaacgtattactgtcatcagagtagtagtttaccgtacacttttggccaggggaccaagctggagatcaaa

SEQ ID NO: 52 - protein, 107 aa, synthetic construct
  REGION 1..107: note = ACRO3-410K
EIVLTQSPDFQSVTPKEKVTITCRASESIGSSLHWYQQKPDQSPKLLIKHASQSFSGVPSRFSGSGSGTDFTLTINGLEAEDAATYYCHQSSSLPYTFGQGTKLEIK

SEQ ID NO: 53 - DNA, 372 bp, synthetic construct
  misc_feature 1..372: note = ACRO3-411L
  source 1..372: mol_type = other DNA
cagctgcagctgcaggagtcgggcccaggactggtgaagccttcggagaccctgtccctcacctgcactgtctctggtggctccatcagcagtgggaattactactggggctggatccgccagcccccagggaaggggctggagtggatcggaaatatctattacagtgggagcacctactacaacccgtccctcaagagtcgagtcaccctctccgtcgacacgcccaagaaccagttctccctgaagctgagctctgtgaccgccgcagacacggctgtgtattactgtgcgagacatgtgttccagctgctattttgggacaactggttcgacccctggggccagggaaccctggtcaccgtctcctca

SEQ ID NO: 54 - protein, 124 aa, synthetic construct
  REGION 1..124: note = ACRO3-411L
QLQLQESGPGLVKPSETLSLTCTVSGGSISSGNYYWGWIRQPPGKGLEWIGNIYYSGSTYYNPSLKSRVTLSVDTPKNQFSLKLSSVTAADTAVYYCARHVFQLLFWDNWFDPWGQGTLVTVSS

SEQ ID NO: 55 - DNA, 330 bp, synthetic construct
  misc_feature 1..330: note = ACRO3-411L
  source 1..330: mol_type = other DNA
cagtctgtgctgactcagccaccctcagcgtctgggacccccgggcagagggtcaccatctcttgttctggaagcagctccaacatcggaagtaatagtgtaaactggtaccagcagatcccaggaccggcccccaaactcctcatccatagtaataatcagcggccctcaggggtccctgaccgattctctggctccaagtcaggcacctcagcctccctgaccatcagcgggatccagtctgaggatgaggctgattattactgtgcagcatgggacgacagcaggaatggtgtggtattcggcggagggaccaagctgaccgtgctg

SEQ ID NO: 56 - protein, 110 aa, synthetic construct
  REGION 1..110: note = ACRO3-411L
QSVLTQPPSASGTPGQRVTISCSGSSSNIGSNSVNWYQQIPGPAPKLLIHSNNQRPSGVPDRFSGSKSGTSASLTISGIQSEDEADYYCAAWDDSRNGVVFGGGTKLTVL

SEQ ID NO: 57 - DNA, 369 bp, synthetic construct
  misc_feature 1..369: note = ACRO3-414K
  source 1..369: mol_type = other DNA
caggtgcagctggtggagtctgggggaggcgtggtccagcctgggaagtccctgagagtctcctgtgcagcctctggattccccttcagtagatttgccttacactgggtccgccaggctccaggcaaggggctggagtgggtggcacttatatcgtttgatggaactaataagttctatgcagactccgtgaagggccgattcaccatctccagagacaattccaagaacattatgtatttggacatgaacagcctgggagacgaagacacggctgtgtattattgtgcgaaagttgttagtggctggcacttagtgacctacggtttggacgtctggggccaagggaccacggtcaccgtctcctca

SEQ ID NO: 58 - protein, 123 aa, synthetic construct
  REGION 1..123: note = ACRO3-414K
QVQLVESGGGVVQPGKSLRVSCAASGFPFSRFALHWVRQAPGKGLEWVALISFDGTNKFYADSVKGRFTISRDNSKNIMYLDMNSLGDEDTAVYYCAKVVSGWHLVTYGLDVWGQGTTVTVSS

SEQ ID NO: 59 - DNA, 336 bp, synthetic construct
  misc_feature 1..336: note = ACRO3-414K
  source 1..336: mol_type = other DNA
gatattgtgatgactcagtctccgctctccctgtccgtcacccctggagagccggcctccatctcttgtaggtcaagtcagagcctcctgcatactaatgggtacgattatttggattggtacctgcagaagccagggcagtctccacagctcctgatgtacttgggttctaatcggggctccggggtccctgtcaggttcagtgccagtggaacaggcacagatttcacactgaaaatcagcagagtggaggctgaggatgttggggtttattactgcatgcaagctctacaaactccgtacacttttggccaggggaccaagctggagatcaaa

SEQ ID NO: 60 - protein, 112 aa, synthetic construct
  REGION 1..112: note = ACRO3-414K
DIVMTQSPLSLSVTPGEPASISCRSSQSLLHTNGYDYLDWYLQKPGQSPQLLMYLGSNRGSGVPVRFSASGTGTDFTLKISRVEAEDVGVYYCMQALQTPYTFGQGTKLEIK

SEQ ID NO: 61 - protein, 5 aa, synthetic construct
  REGION 1..5: note = ACRO2-001K
ANYMS

SEQ ID NO: 62 - protein, 21 aa, synthetic construct
  REGION 1..21: note = ACRO2-001K
VIYSGGSTNDGDTYYAASVKG

SEQ ID NO: 63 - protein, 8 aa, synthetic construct
  REGION 1..8: note = ACRO2-001K
VVSDAFDI

SEQ ID NO: 64 - protein, 11 aa, synthetic construct
  REGION 1..11: note = ACRO2-001K
RASQSISSWLA

SEQ ID NO: 65 - protein, 7 aa, synthetic construct
  REGION 1..7: note = ACRO2-001K
KASILES

SEQ ID NO: 66 - protein, 9 aa, synthetic construct
  REGION 1..9: note = ACRO2-001K
QHYNSDSYT

SEQ ID NO: 67 - protein, 5 aa, synthetic construct
  REGION 1..5: note = ACRO2-021L
SFAMT

SEQ ID NO: 68 - protein, 17 aa, synthetic construct
  REGION 1..17: note = ACRO2-021L
VISGGAGSTYYADSVKG

SEQ ID NO: 69 - protein, 12 aa, synthetic construct
  REGION 1..12: note = ACRO2-021L
GERPDYGDYFDY

SEQ ID NO: 70 - protein, 11 aa, synthetic construct
  REGION 1..11: note = ACRO2-021L
GGNNIGSKSVH

SEQ ID NO: 71 - protein, 7 aa, synthetic construct
  REGION 1..7: note = ACRO2-021L
YDSDRPS

SEQ ID NO: 72 - protein, 12 aa, synthetic construct
  REGION 1..12: note = ACRO2-021L
QVWDGSSDHHYV

SEQ ID NO: 73 - protein, 5 aa, synthetic construct
  REGION 1..5: note = ACRO2-028K
SNYMS

SEQ ID NO: 74 - protein, 16 aa, synthetic construct
  REGION 1..16: note = ACRO2-028K
VIYSGGSTYYADSVKG

SEQ ID NO: 75 - protein, 8 aa, synthetic construct
  REGION 1..8: note = ACRO2-028K
VVTDAFDI

SEQ ID NO: 76 - protein, 11 aa, synthetic construct
  REGION 1..11: note = ACRO2-028K
RASQSISSWLA

SEQ ID NO: 77 - protein, 7 aa, synthetic construct
  REGION 1..7: note = ACRO2-028K
KASSLES

SEQ ID NO: 78 - protein, 9 aa, synthetic construct
  REGION 1..9: note = ACRO2-028K
QQYNSDSYT

SEQ ID NO: 79 - protein, 7 aa, synthetic construct
  REGION 1..7: note = ACRO3-168L
SSRYYWG

SEQ ID NO: 80 - protein, 16 aa, synthetic construct
  REGION 1..16: note = ACRO3-168L
SLYYSGGTYFNPSLKS

SEQ ID NO: 81 - protein, 10 aa, synthetic construct
  REGION 1..10: note = ACRO3-168L
DHYGDYALVY

SEQ ID NO: 82 - protein, 14 aa, synthetic construct
  REGION 1..14: note = ACRO3-168L
TGSSSNIGAGYDVH

SEQ ID NO: 83 - protein, 7 aa, synthetic construct
  REGION 1..7: note = ACRO3-168L
GHNNRPS

SEQ ID NO: 84 - protein, 12 aa, synthetic construct
  REGION 1..12: note = ACRO3-168L
QSYDTSLSALYV

SEQ ID NO: 85 - protein, 7 aa, synthetic construct
  REGION 1..7: note = ACRO3-169L
SSRYYWG

SEQ ID NO: 86 - protein, 16 aa, synthetic construct
  REGION 1..16: note = ACRO3-169L
SLYYSGGTYYNPSLKS

SEQ ID NO: 87 - protein, 10 aa, synthetic construct
  REGION 1..10: note = ACRO3-169L
DHYGDYALVY

SEQ ID NO: 88 - protein, 14 aa, synthetic construct
  REGION 1..14: note = ACRO3-169L
TGSSSNVGAGYDVH

SEQ ID NO: 89 - protein, 7 aa, synthetic construct
  REGION 1..7: note = ACRO3-169L
GNNNRPS

SEQ ID NO: 90 - protein, 12 aa, synthetic construct
  REGION 1..12: note = ACRO3-169L
QSYDTSLSALYV

SEQ ID NO: 91 - protein, 5 aa, synthetic construct
  REGION 1..5: note = ACRO3-207L
SYAMN

SEQ ID NO: 92 - protein, 17 aa, synthetic construct
  REGION 1..17: note = ACRO3-207L
WINTNTGNPTYAQGFTG

SEQ ID NO: 93 - protein, 9 aa, synthetic construct
  REGION 1..9: note = ACRO3-207L
ETSWDGYIY

SEQ ID NO: 94 - protein, 11 aa, synthetic construct
  REGION 1..11: note = ACRO3-207L
SGDTLSKQYTF

SEQ ID NO: 95 - protein, 7 aa, synthetic construct
  REGION 1..7: note = ACRO3-207L
KDSERPS

SEQ ID NO: 96 - protein, 11 aa, synthetic construct
  REGION 1..11: note = ACRO3-207L
QSADNSGTSWV

SEQ ID NO: 97 - protein, 5 aa, synthetic construct
  REGION 1..5: note = ACRO3-283L
ELSMH

SEQ ID NO: 98 - protein, 17 aa, synthetic construct
  REGION 1..17: note = ACRO3-283L
GFDPEDAETIYAQKFQG

SEQ ID NO: 99 - protein, 14 aa, synthetic construct
  REGION 1..14: note = ACRO3-283L
GPAIPMAATGWFDP

SEQ ID NO: 100 - protein, 14 aa, synthetic construct
  REGION 1..14: note = ACRO3-283L
TGTSSDVGGYNYVS

SEQ ID NO: 101 - protein, 7 aa, synthetic construct
  REGION 1..7: note = ACRO3-283L
EVSNRPS

SEQ ID NO: 102 - protein, 10 aa, synthetic construct
  REGION 1..10: note = ACRO3-283L
SSYTSSSTYV

SEQ ID NO: 103 - protein, 5 aa, synthetic construct
  REGION 1..5: note = ACRO3-286L
SYYIH

SEQ ID NO: 104 - protein, 17 aa, synthetic construct
  REGION 1..17: note = ACRO3-286L
IINPGDGGASYAQKFQG

SEQ ID NO: 105 - protein, 12 aa, synthetic construct
  REGION 1..12: note = ACRO3-286L
AEGSSWLGWFDP

SEQ ID NO: 106 - protein, 13 aa, synthetic construct
  REGION 1..13: note = ACRO3-286L
SGSSSNIGSNYVY

SEQ ID NO: 107 - protein, 7 aa, synthetic construct
  REGION 1..7: note = ACRO3-286L
RNNQRPS

SEQ ID NO: 108 - protein, 13 aa, synthetic construct
  REGION 1..13: note = ACRO3-286L
AAWDDGLSGSGWV

SEQ ID NO: 109 - protein, 5 aa, synthetic construct
  REGION 1..5: note = ACRO3-307K
NYAMH

SEQ ID NO: 110 - protein, 17 aa, synthetic construct
  REGION 1..17: note = ACRO3-307K
VIWDDGSNKHYADSVKG

SEQ ID NO: 111 - protein, 11 aa, synthetic construct
  REGION 1..11: note = ACRO3-307K
DQGELVTYFDY

SEQ ID NO: 112 - protein, 11 aa, synthetic construct
  REGION 1..11: note = ACRO3-307K
RASQTISSYLN

SEQ ID NO: 113 - protein, 7 aa, synthetic construct
  REGION 1..7: note = ACRO3-307K
TTSSLQS

SEQ ID NO: 114 - protein, 10 aa, synthetic construct
  REGION 1..10: note = ACRO3-307K
QQSYSTPSWT

SEQ ID NO: 115 - protein, 5 aa, synthetic construct
  REGION 1..5: note = ACRO3-309K
NYDIH

SEQ ID NO: 116 - protein, 16 aa, synthetic construct
  REGION 1..16: note = ACRO3-309K
CVGTYGDTYYSDSVKG

SEQ ID NO: 117 - protein, 13 aa, synthetic construct
  REGION 1..13: note = ACRO3-309K
GSVDPTTNWFFDL

SEQ ID NO: 118 - protein, 11 aa, synthetic construct
  REGION 1..11: note = ACRO3-309K
RASQGIRNFLA

SEQ ID NO: 119 - protein, 7 aa, synthetic construct
  REGION 1..7: note = ACRO3-309K
AASRLES

SEQ ID NO: 120 - protein, 10 aa, synthetic construct
  REGION 1..10: note = ACRO3-309K
QQYYSSPPIT

SEQ ID NO: 121 - protein, 5 aa, synthetic construct
  REGION 1..5: note = ACRO3-341L
SYSMN

SEQ ID NO: 122 - protein, 17 aa, synthetic construct
  REGION 1..17: note = ACRO3-341L
YISSSNITIYYADSVKG

SEQ ID NO: 123 - protein, 21 aa, synthetic construct
  REGION 1..21: note = ACRO3-341L
VLRYYDYVWGSYQRELYGMDV

SEQ ID NO: 124 - protein, 14 aa, synthetic construct
  REGION 1..14: note = ACRO3-341L
TGTSSDVGGYDYVS

SEQ ID NO: 125 - protein, 7 aa, synthetic construct
  REGION 1..7: note = ACRO3-341L
DVSNRPS

SEQ ID NO: 126 - protein, 12 aa, synthetic construct
  REGION 1..12: note = ACRO3-341L
SSFTSSSTLPFV

SEQ ID NO: 127 - protein, 6 aa, synthetic construct
  REGION 1..6: note = ACRO3-347K
DVSSLQ

SEQ ID NO: 128 - protein, 17 aa, synthetic construct
  REGION 1..17: note = ACRO3-347K
WTVVGTGNTNYAPRFQE

SEQ ID NO: 129 - protein, 14 aa, synthetic construct
  REGION 1..14: note = ACRO3-347K
PFCSETSCSDGFDL

SEQ ID NO: 130 - protein, 12 aa, synthetic construct
  REGION 1..12: note = ACRO3-347K
RASQSVRISYLA

SEQ ID NO: 131 - protein, 7 aa, synthetic construct
  REGION 1..7: note = ACRO3-347K
GSSSRAT

SEQ ID NO: 132 - protein, 9 aa, synthetic construct
  REGION 1..9: note = ACRO3-347K
QQYDNSPWT

SEQ ID NO: 133 - protein, 5 aa, synthetic construct
  REGION 1..5: note = ACRO3-410K
NYGIS

SEQ ID NO: 134 - protein, 17 aa, synthetic construct
  REGION 1..17: note = ACRO3-410K
WISSYNGNTHYARKLQG

SEQ ID NO: 135 - protein, 14 aa, synthetic construct
  REGION 1..14: note = ACRO3-410K
ENFNYGDYIIYFDP

SEQ ID NO: 136 - protein, 11 aa, synthetic construct
  REGION 1..11: note = ACRO3-410K
RASESIGSSLH

SEQ ID NO: 137 - protein, 7 aa, synthetic construct
  REGION 1..7: note = ACRO3-410K
HASQSFS

SEQ ID NO: 138 - protein, 9 aa, synthetic construct
  REGION 1..9: note = ACRO3-410K
HQSSSLPYT

SEQ ID NO: 139 - protein, 7 aa, synthetic construct
  REGION 1..7: note = ACRO3-411L
SGNYYWG

SEQ ID NO: 140 - protein, 16 aa, synthetic construct
  REGION 1..16: note = ACRO3-411L
NIYYSGSTYYNPSLKS

SEQ ID NO: 141 - protein, 14 aa, synthetic construct
  REGION 1..14: note = ACRO3-411L
HVFQLLFWDNWFDP

SEQ ID NO: 142 - protein, 13 aa, synthetic construct
  REGION 1..13: note = ACRO3-411L
SGSSSNIGSNSVN

SEQ ID NO: 143 - protein, 7 aa, synthetic construct
  REGION 1..7: note = ACRO3-411L
SNNQRPS

SEQ ID NO: 144 - protein, 11 aa, synthetic construct
  REGION 1..11: note = ACRO3-411L
AAWDDSRNGVV

SEQ ID NO: 145 - protein, 5 aa, synthetic construct
  REGION 1..5: note = ACRO3-414K
RFALH

SEQ ID NO: 146 - protein, 17 aa, synthetic construct
  REGION 1..17: note = ACRO3-414K
LISFDGTNKFYADSVKG

SEQ ID NO: 147 - protein, 14 aa, synthetic construct
  REGION 1..14: note = ACRO3-414K
VVSGWHLVTYGLDV

SEQ ID NO: 148 - protein, 16 aa, synthetic construct
  REGION 1..16: note = ACRO3-414K
RSSQSLLHTNGYDYLD

SEQ ID NO: 149 - protein, 7 aa, synthetic construct
  REGION 1..7: note = ACRO3-414K
LGSNRGS

SEQ ID NO: 150 - protein, 9 aa, synthetic construct
  REGION 1..9: note = ACRO3-414K
MQALQTPYT